(19) United States
(12) Patent Application Publication
(10) Pub. No.: US 2006/0099599 A1
(43) Pub. Date: May 11, 2006

(54) Role of Glycogen Synthase Kinase-3 and tetraspanins in ethanol-induced henaviors

(75) Inventors: Ulrike Heberlein, Hillsborough, CA (US); Fred Wolf, San Francisco, CA (US)

Correspondence Address: QUINE INTELLECTUAL PROPERTY LAW GROUP, P.C., P O BOX 458, ALAMEDA, CA, 94501, US

(73) Assignee: The Reagens of the University of California

(21) Appl. No.: 10/795,135

(22) Filed: Mar. 5, 2004

Related U.S. Application Data
(60) Provisional application No. 60/452,486, filed on Mar. 5, 2003.

(51) Int. Cl. C12Q 1/68

(52) U.S. Cl. 435/6

(57) ABSTRACT

This invention pertains to the identification of genes that mediate an organisms behavioral response to consumption of alcohol and/or other substances of abuse. The genes include a tetraspanin Tsp42Ee gene or a homologue or analogue thereof, a tetraspanin Tsp42El gene or a homologue or analogue thereof, and a Glycogen Synthase Kinase-3 gene or a homologue or analogue thereof. The genes provide good targets to screen for agents that modulate an organism's response to consumption of alcohol and/or other substances of abuse.

### CROSS-REFERENCE TO RELATED APPLICATIONS

This application claims benefit of and priority to U.S. Ser. No. 60/452,486, filed on Mar. 5, 2003, which is incorporated herein by reference in its entirety for all purposes.

### STATEMENT AS TO RIGHTS TO INVENTIONS MADE UNDER FEDERALLY SPONSORED RESEARCH AND DEVELOPMENT

This work was supported by a grant from the National Institutes of Health National Institute on Alcohol Abuse and Alcoholism. The Government of the United States of America may have certain rights in this invention.

### FIELD OF THE INVENTION

This invention pertains to the field of neurobiology and substance abuse. In particular, this invention pertains to the identification of genes that regulate acute ethanol-induced behaviors.

### BACKGROUND OF THE INVENTION

Ethyl alcohol (ethanol) is the most widely used psychoactive drug in the world. Alcohol abuse and alcohol related diseases represent a serious threat to human health and pose major medical, social, and economic problems. In the United States alone, an estimated 10% of the population is affected by alcoholism. The problems associated with alcohol abuse are costly, both to the individuals affected and to society at large. The physical, social and psychological harm that can result from alcohol abuse and dependence, such as fetal alcohol syndrome, cirrhosis of the liver, alcohol-related accidental death, homicide, suicide, etc., can be devastating. Thus, there remains a strong need to develop safe and effective therapeutic agents for treating alcohol abuse and dependence.

As public awareness of the problems associated with alcohol abuse has increased in recent years, greater efforts have been devoted to the development of treatments for alcoholism. Much of the current research in this area has focused on methods for treating the effects of alcohol withdrawal through the clinical use of various drugs, such as benzodiazepines and the antidipsotropic agent disulfuram (Antabuse™). Recent research has also led to the development of new therapeutic agents which suppress alcohol drinking in humans. For instance, dopamine agonists and antagonists, serotonergic agents, glutamate antagonists, opiate antagonists, ALDH inhibitors, and calcium blockers have been reported to reduce self administration of alcohol in alcoholic humans and alcohol-preferring rats. Unfortunately, many of these current drug treatments are toxic, exhibit low efficacy and patient compliance, have many adverse side effect, and are unsuitable for use with adolescents and pregnant women. Thus, despite these recent advancements, developing effective treatments for alcohol dependence remains a challenging goal.

### SUMMARY OF THE INVENTION

This invention pertains to the identification of genes that mediate an organisms behavioral response to consumption of alcohol and/or other substances of abuse. The genes include a tetraspanin Tsp42Ee gene or a homologue or analogue thereof, a tetraspanin Tsp42El gene or a homologue or analogue thereof, and a Glycogen Synthase Kinase-3 gene or a homologue or analogue thereof. The genes provide good targets to screen for agents that modulate an organism's response to consumption of alcohol and/or other substances of abuse.

Thus, in one embodiment, this invention provides a method of identifying an agent that modulates a behavioral response to consumption or ethanol and/or other substances of abuse. The method typically involves contacting a cell or a tissue with a test agent; detecting expression or activity of a gene selected from the group consisting of a tetraspanin Tsp42Ee gene or a homologue or analogue thereof, a tetraspanin Tsp42El gene or a homologue or analogue thereof, and a Glycogen Synthase Kinase-3 gene or a homologue or analogue thereof; where a change in activity or expression of the factor, as compared to a cell or tissue that is a control indicates that said test agent is a good candidate for modulating a behavioral response to consumption or ethanol and/or other substances of abuse. In certain embodiments, the cell or tissue is a neural cell or tissue. In certain embodiments, the tetraspanin Tsp42Ee homologue or analogue and/or the tetraspanin Tsp42El homologue or analogue and/or the Glycogen Synthase Kinase-3 homologue or analogue is a human homologue or analogue. The control can comprise a negative control comprising a cell or tissue contacted with the test agent at a lower concentration. In certain embodiments, the control is a negative control comprising a cell or tissue not contacted with said test agent. In certain embodiments, the control is a positive control comprising a cell or tissue contacted with said test agent at a higher concentration. In various embodiments, the detecting comprises detecting a tetraspanin Tsp42Ee mRNA, a Tsp42El mRNA, or a Glycogen Synthase Kinase-3 mRNA and/or reverse transcribed cDNA. In certain embodiments, the level of tetraspanin Tsp42Ee mRNA, Tsp42El mRNA, or Glycogen Synthase Kinase-3 mRNA is measured by hybridizing said mRNA to a probe that specifically hybridizes to a tetraspanin Tsp42Ee nucleic acid, a Tsp42El nucleic acid, or a Glycogen Synthase Kinase-3 nucleic acid (e.g. under stringent conditions). In various embodiments, the hybridizing is according to a method selected from the group consisting of a Northern blot, a Southern blot using DNA derived from the tetraspanin Tsp42Ee mRNA, Tsp42El mRNA, or Glycogen Synthase Kinase-3 mRNA, an array hybridization, an affinity chromatography, and an in situ hybridization. In certain embodiments, the probe is a member of a plurality of probes that forms an array of probes (e.g. in a high-density array). In certain embodiments, the level of tetraspanin Tsp42Ee mRNA, Tsp42El mRNA, or Glycogen Synthase Kinase-3 mRNA is measured using a nucleic acid amplification reaction. In certain embodiments, the detecting comprises detecting a tetraspanin Tsp42Ee protein, a Tsp42El protein, and/or Glycogen Synthase Kinase-3 protein (e.g., via capillary electrophoresis, a Western blot, mass spectroscopy, ELISA, immunochromatography, immunohistochemistry, etc.). The cell can be a cell grown in vivo or cultured ex vivo. In certain embodiments, the test agent is contacted to a mammal comprising said cell or tissue.

In another embodiment, this invention provides a method of prescreening for an agent that modulates a behavioral response to consumption or ethanol or other substances of abuse. The method typically involves contacting a gene or gene product from a gene selected from the group consisting of a tetraspanin Tsp42Ee gene or a homologue or analogue thereof, a tetraspanin Tsp42El gene or a homologue or analogue thereof, and a Glycogen Synthase Kinase-3 gene or a homologue or analogue thereof with a test agent; and detecting specific binding of the test agent to the gene or gene product, where specific binding indicates that the agent is a candidate modulator of a behavioral response to consumption of ethanol or other substances of abuse. In certain embodiments, the homologue or analogue is a human homologue or analogue. The method can, optionally, further comprise recording test agents that specifically bind to said gene or gene product, in a database of candidate agents that modulate an organisms behavioral response to ethanol consumption. In certain embodiments, the test agent is not an antibody and/or the test agent is not a protein, and/or the test agent is not a nucleic acid. In certain preferred embodiments the test agent is a small organic molecule. The detecting can comprise detecting specific binding of the test agent to a tetraspanin Tsp42Ee nucleic acid, and/or to a Tsp42El nucleic acid, and/or to a Glycogen Synthase Kinase-3 nucleic acid (e.g. via a Northern blot, a Southern blot using DNA derived from a tetraspanin Tsp42Ee gene, a tetraspanin Tsp42El gene, and/or a Glycogen Synthase Kinase-3 gene, an array hybridization, an affinity chromatography, an in situ hybridization, etc.). In certain embodiments, the detecting comprises detecting specific binding of the test agent to a tetraspanin Tsp42Ee protein, and/or to a Tsp42El protein, and/or to a Glycogen Synthase Kinase-3 protein (e.g. via capillary electrophoresis, a Western blot, mass spectroscopy, ELISA, immunochromatography, immunohistochemistry. gel shift assay, etc.). In various embodiments, the test agent is contacted directly to the gene or gene product, and/or to a cell containing gene or gene product and/or to an animal comprising such a cell.

Also provided is a method of altering the behavioral response of an organism consumption of ethanol and/or other substances of abuse. The method typically involves altering expression or activity of a gene selected from the group consisting of a tetraspanin Tsp42Ee gene or a homologue or analogue thereof, a tetraspanin Tsp42El gene or a homologue or analogue thereof, and a Glycogen Synthase Kinase-3 gene or a homologue or analogue thereof. In certain embodiments, the altering comprises increasing or decreasing the expression or activity of the gene(s).

In still another embodiment, this invention provides an antibody that specifically binds to a gene product from a gene selected from the group consisting of a tetraspanin Tsp42Ee gene or a homologue or analogue thereof, a tetraspanin Tsp42El gene or a homologue or analogue thereof, and a Glycogen Synthase Kinase-3 gene or a homologue or analogue thereof. In certain embodiments, the antibody is a monoclonal antibody, a polyclonal antibody, an antibody fragment, a single chain antibody, and the like.

In certain embodiments, a knockout animal is also provided. The animal typically comprises disruption in one or more endogenous gene(s) selected from the group consisting of a tetraspanin Tsp42Ee gene or a homologue or analogue thereof, a tetraspanin Tsp42El gene or a homologue or analogue thereof, and a Glycogen Synthase Kinase-3 gene or a homologue or analogue thereof. In certain preferred embodiments, the animal shows an altered response to consumption of alcohol or other substances of abuse as compared to a wild-type animal. In certain embodiments, the animal is a non-human mammal (e.g. an equine, a bovine, a rodent, a porcine, a lagomorph, a feline, a canine, a murine, an ovine, a non-human primate, etc.). In certain embodiments, the disruption is an insertion, a deletion, a frameshift mutation, a substitution, or the insertion of a stop codon. In certain embodiments, the disruption comprises an insertion of an expression cassette into the endogenous gene. Certain preferred cassettes comprise a selectable marker (e.g., a neomycin phosphotransferase gene operably linked to at least one regulatory element). In certain embodiments, the disruption is in a somatic cell and/or a germ cell. The mammal can be homozygous, heterozygous or chimeric for the disrupted gene.

Definitions

The term “substance of abuse” refers to a substance that is psychoactive and that induces tolerance and/or addiction. Substances of abuse include, but are not limited to stimulants (e.g. cocaine, amphetamines), opiates (e.g. morphine, heroin), cannabinoids (e.g. marijuana, hashish), nicotine, alcohol, substances that mediate agonist activity at the dopamine D2 receptor, and the like. Substances of abuse include, but are not limited to addictive drugs.

The term “gene product” refers to a molecule that is ultimately derived from a gene. The molecule can be a polypeptide encoded by the gene, an mRNA encoded by a gene, a cDNA reverse transcribed from the mRNA, and so forth.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

The term “neurotrophic and/or neurogenerative” factor refers to an agent that induces migration of a cell to a neural tissue (neurotrophic) and/or that induces growth or differentiation of a neural cell or tissue.

The term “antibody”, as used herein, includes various forms of modified or altered antibodies, such as an intact immunoglobulin, an Fv fragment containing only the light and heavy chain variable regions, an Fv fragment linked by a disulfide bond (Brinkmann et al. (1993) Proc. Natl. Acad. Sci. USA, 90: 547-551), an Fab or (Fab)′2 fragment containing the variable regions and parts of the constant regions, a single-chain antibody and the like (Bird et al. (1988) Science 242: 424-426; Huston et al. (1988) Proc. Nat. Acad. Sci. USA 85: 5879-5883). The antibody may be of animal (especially mouse or rat) or human origin or may be chimeric (Morrison et al. (1984) Proc Nat. Acad. Sci. USA 81: 6851-6855) or humanized (Jones et al. (1986) Nature 321: 522-525, and published UK patent application #8707252).

The terms “binding partner”, or “capture agent”, or a member of a “binding pair” refers to molecules that specifically bind other molecules to form a binding complex such as antibody-antigen, lectin-carbohydrate, nucleic acid-nucleic acid, biotin-avidin, etc.

The term “specifically binds”, as used herein, when referring to a biomolecule (e.g., protein, nucleic acid, antibody, etc.), refers to a binding reaction which is determinative of the presence biomolecule in heterogeneous population of molecules (e.g., proteins and other biologics). Thus, under designated conditions (e.g. immunoassay conditions in the case of an antibody or stringent hybridization conditions in the case of a nucleic acid), the specified ligand or antibody binds to its particular “target” molecule and does not bind in a significant amount to other molecules present in the sample.

The terms “nucleic acid” or “oligonucleotide” or grammatical equivalents herein refer to at least two nucleotides covalently linked together. A nucleic acid of the present invention is preferably single-stranded or double stranded and will generally contain phosphodiester bonds, although in some cases, as outlined below, nucleic acid analogs are included that may have alternate backbones, comprising, for example, phosphoramide (Beaucage et al. (1993) Tetrahedron 49(10): 1925) and references therein; Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 141 9), phosphorothioate (Mag et al. (1991) Nucleic Acids Res. 19:1437; and U.S. Pat. No. 5,644,048), phosphorodithioate (Briu et al. (1989) J. Am. Chem. Soc. 111:2321, O-methylphophoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Angew. (1991) Chem. Intl. Ed. English 30: 423; Letsinger et al. (1988) J. Am. Chem. Soc. 110:4470; Letsinger et al. (1994) Nucleoside &Nucleotide 13:1597; Chapters 2 and 3, ASC Symposium Series 580, “Carbohydrate Modifications in Antisense Research”, Ed. Y. S. Sanghui and P. Dan Cook; Mesmaeker et al. (1994), Bioorganic &Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp 169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions. The term “stringent conditions” refers to conditions under which a probe will hybridize preferentially to its target subsequence, and to a lesser extent to, or not at all to, other sequences. Stringent hybridization and stringent hybridization wash conditions in the context of nucleic acid hybridization are sequence dependent, and are different under different environmental parameters. An extensive guide to the hybridization of nucleic acids is found in, e.g., Tijssen (1993) Laboratory Techniques in Biochemistry and Molecular Biology—Hybridization with Nucleic Acid Probes part I, chapt 2, Overview of principles of hybridization and the strategy of nucleic acid probe assays, Elsevier, N.Y. (Tijssen). Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on an array or on a filter in a Southern or northern blot is 42° C. using standard hybridization solutions (see, e.g., Sambrook (1989) Molecular Cloning: A Laboratory Manual (2nd ed.) Vol. 1-3, Cold Spring Harbor Laboratory, Cold Spring Harbor Press, NY, and detailed discussion, below), with the hybridization being carried out overnight. An example of highly stringent wash conditions is 0.15 M NaCl at 72° C. for about 15 minutes. An example of stringent wash conditions is a 0.2×SSC wash at 65° C. for 15 minutes (see, e.g., Sambrook supra.) for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example of a low stringency wash for a duplex of, e.g., more than 100 nucleotides, is 4× to 6×SSC at 40° C. for 15 minutes.

The term “test agent” refers to an agent that is to be screened in one or more of the assays described herein. The agent can be virtually any chemical compound. It can exist as a single isolated compound or can be a member of a chemical (e.g. combinatorial) library. In a particularly preferred embodiment, the test agent will be a small organic molecule.

The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

The term database refers to a means for recording and retrieving information. In preferred embodiments the database also provides means for sorting and/or searching the stored information. The database can comprise any convenient media including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems”, mainframe systems, distributed nodes on an inter- or intra-net, data or databases stored in specialized hardware (e.g. in microchips), and the like.

The phrase “expression or activity of a gene” (e.g. Tsp42Ee gene) refers to the production of a gene product (e.g. the production of an mRNA and/or a protein) or to the activity of a gene product (i.e., the activity of a protein encoded by the gene).

The term “expression” refers to protein expression, e.g., mRNA and/or translation into protein. The term “activity” refers to the activity of a protein. Activities include but are not limited to phosphorylation, signaling activity, activation, catalytic activity, protein-protein interaction, transportation, etc. The expression and/or activity can increase, or decrease. Expression and/or activity can be activated or inhibited directly or indirectly.

A “Tsp42Ee, a Tsp42El and/or a GSK-3 nucleotide or polypeptide” when used with respect to a mouse or other organism (other than Drosophila) refers to the Tsp42Ee, a Tsp42El and/or a GSK-3 homologue found in the mouse or other organism.

### BRIEF DESCRIPTION OF THE DRAWINGS

[Not Applicable]

### DETAILED DESCRIPTION

Understanding of how ethanol influences behavior is key to deciphering the mechanisms of ethanol action and alcoholism. In mammals, low doses of ethanol stimulate locomotion, whereas high doses depress it. The acute stimulant effect of ethanol has been proposed to be a manifestation of its rewarding effects. In Drosophila, ethanol exposure transiently potentiates locomotor activity in a biphasic dose- and time-dependent manner. An initial short-lived peak of activity corresponds to an olfactory response to ethanol. A second, longer-lasting period of increased activity coincides with rising internal ethanol concentrations; these closely parallel concentrations that stimulate locomotion in mammals.

High-resolution analysis of the walking pattern of individual flies revealed that locomotion consists of bouts of activity; bout structure can be quantified by bout frequency, bout length, and the time spent walking at high speeds. Ethanol exposure induces both dramatic and dynamic changes in bout structure. Mutants with increased ethanol sensitivity show distinct changes in ethanol-induced locomotor behavior, as well as genotype-specific changes in activity bout structure. Thus, the overall effect of ethanol on locomotor behavior in Drosophila is caused by changes in discrete quantifiable parameters of walking pattern. The effects of ethanol on locomotion are comparable in flies and mammals, indicating that Drosophila is a suitable model system to study the underlying mechanisms.

This invention pertains to the identification of particular genes that mediate a behavioral response to acute treatment with ethanol, by implication, mediate a behavioral response to other substances of abuse (e.g. cocaine, heroin, marijuana, nicotine, and the like). The genes, originally identified in Drosophila include Tsp42Ee, encoding a putative tetraspanin, carrying the following name according to the published Drosophila genome sequence: CG10106, Tsp42El, encoding a putative tetraspanin, carrying the following name according to the published Drosophila genome sequence: CG12840, and shaggy (sgg), encoding Glycogen Synthase Kinase 3 (GSK3), a protein serine/threonine kinase, carrying following name according to the published Drosophila genome sequence: CG2621. Homologues and analogues of these genes (e.g. human homologues or analogues) are believed to act in a similar manner and to mediate the response of an organism to consumption of ethanol and/or other substances of abuse.

These genes and their homologues and analogues provide good targets to screen for agents that modulate the response of a organism (e.g. a human or non-human mammal) to consumption of ethanol and/or other substances of abuse.

Thus, in one embodiment, this invention provides methods of screening for modulators (e.g. upregulators and/or downregulators) of these genes. Also provided are antibodies to the genes or gene products that are useful in such assays.

In addition, this invention provides methods of modulating the response of an organism to consumption of ethanol or other substances of abuse by modulating the expression and/or activity of these genes or their gene products.

I. Assays for Agents that Modulate an Organisms Behavioral Response to Consumption of Alcohol or Other Substances of Abuse.

As indicated above, in one aspect, this invention is premised on the discovery that Tsp42Ee genes (e.g. Tsp42Ee and its homologues or analogues), Tsp42El genes ((e.g. Tsp42El and its homologues or analogues), and shaggy (sgg) effect an organism's behavioral response to consumption of alcohol and/or other substances of abuse. Thus, agents that modulate (e.g., upregulate and/or downregulate) the expression and/or activity of these genes are expected to have prophylactic and/or therapeutic utility in treatment of substance abuse. Thus, in one embodiment, this invention provides methods of screening for agents that modulate expression or activity of these genes or gene products

The methods typically involve detecting alterations in the expression level and/or activity level of a Tsp42Ee gene or gene product, and/or a Tsp42El gene or gene product, and/or an sgg gene or gene product caused by the treatment with one or more of the agent(s) in question. An elevated expression level or activity level produced by the agent as, e.g., compared to a negative control where the test agent is absent or at reduced concentration indicates that the agent upregulates activity or expression of the factor(s) in question. Conversely, decreased expression level or activity level resulting from treatment by the agent as compared to a negative control where the test agent is absent or at reduced concentration indicates that the agent down-regulates expression or activity of the factor(s).

Expression levels of a gene can be altered by changes in by changes in the transcription of the gene product (i.e. transcription of mRNA), and/or by changes in translation of the gene product (i.e. translation of the protein), and/or by post-translational modification(s) (e.g. protein folding, glycosylation, etc.). Thus preferred assays of this invention typically contacting a test cell, tissue, or animal with one or more test agents, and assaying for level of transcribed mRNA (or other nucleic acids derived from the neurotrophic and/or neurogenerative factor gene(s)), level of translated protein, activity of translated protein, etc. Examples of such approaches are described below.

A) Nucleic-Acid Based Assays.

1) Target Molecules.

Changes in expression level can be detected by measuring changes in mRNA and/or a nucleic acid derived from the mRNA (e.g. reverse-transcribed cDNA, etc.). In order to measure the Tsp42Ee, and/or a Tsp42El, and/or an sgg expression level it is desirable to provide a nucleic acid sample for such analysis. In preferred embodiments the nucleic acid is found in or derived from a biological sample. The term “biological sample”, as used herein, refers to a sample obtained from an organism or from components (e.g., cells) of an organism. The sample may be of any biological tissue or fluid. Biological samples may also include organs or sections of tissues such as frozen sections taken for histological purposes.

The nucleic acid (e.g., mRNA nucleic acid derived from mRNA) is, in certain preferred embodiments, isolated from the sample according to any of a number of methods well known to those of skill in the art. Methods of isolating mRNA are well known to those of skill in the art. For example, methods of isolation and purification of nucleic acids are described in detail in by Tijssen ed., (1993) Chapter 3 of Laboratory Techniques in Biochemistry and Molecular Biology: Hybridization With Nucleic Acid Probes, Part I. Theory and Nucleic Acid Preparation, Elsevier, N.Y. and Tijssen ed.

In a preferred embodiment, the “total” nucleic acid is isolated from a given sample using, for example, an acid guanidinium-phenol-chloroform extraction method and polyA+ mRNA is isolated by oligo dT column chromatography or by using (dT)n magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)).

Frequently, it is desirable to amplify the nucleic acid sample prior to assaying for expression level. Methods of amplifying nucleic acids are well known to those of skill in the art and include, but are not limited to polymerase chain reaction (PCR, see. e.g., Innis, et al., (1990) PCR Protocols. A guide to Methods and Application. Academic Press, Inc. San Diego,), ligase chain reaction (LCR) (see Wu and Wallace (1989) Genomics 4: 560, Landegren et al. (1988) Science 241: 1077, and Barringer et al. (1990) Gene 89: 117, transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874), dot PCR, and linker adapter PCR, etc.).

In a particularly preferred embodiment, where it is desired to quantify the transcription level (and thereby expression) of factor(s) of interest in a sample, the nucleic acid sample is one in which the concentration of the Tsp42Ee, and/or a Tsp42El, and/or an sgg transcript(s), or the concentration of the nucleic acids derived from the Tsp42Ee, and/or a Tsp42El, and/or an sgg mRNA transcript(s), is proportional to the transcription level (and therefore expression level) of that gene. Similarly, it is preferred that the hybridization signal intensity be proportional to the amount of hybridized nucleic acid. While it is preferred that the proportionality be relatively strict (e.g., a doubling in transcription rate results in a doubling in mRNA transcript in the sample nucleic acid pool and a doubling in hybridization signal), one of skill will appreciate that the proportionality can be more relaxed and even non-linear. Thus, for example, an assay where a 5 fold difference in concentration of the target mRNA results in a 3 to 6 fold difference in hybridization intensity is sufficient for most purposes.

Where more precise quantification is required, appropriate controls can be run to correct for variations introduced in sample preparation and hybridization as described herein. In addition, serial dilutions of “standard” target nucleic acids (e.g., mRNAs) can be used to prepare calibration curves according to methods well known to those of skill in the art. Of course, where simple detection of the presence or absence of a transcript or large differences of changes in nucleic acid concentration is desired, no elaborate control or calibration is required.

In the simplest embodiment, the nucleic acid sample is the total mRNA or a total cDNA isolated and/or otherwise derived from a biological sample (e.g. a sample from a neural cell or tissue). The nucleic acid may be isolated from the sample according to any of a number of methods well known to those of skill in the art as indicated above.

2) Hybridization-Based Assays.

Using the known sequence of Tsp42Ee, and/or Tsp42El, and/or sgg (see, e.g., Examples 1-3) detecting and/or quantifying the transcript(s) can be routinely accomplished using nucleic acid hybridization techniques (see, e.g., Sambrook et al. supra). For example, one method for evaluating the presence, absence, or quantity of reverse-transcribed cDNA involves a “Southern Blot”. In a Southern Blot, the DNA (e.g., reverse-transcribed Tsp42Ee, and/or Tsp42El, and/or sgg mRNA), typically fragmented and separated on an electrophoretic gel, is hybridized to a probe specific for the nucleic acid encoding the Tsp42Ee, and/or Tsp42El, and/or sgg. Comparison of the intensity of the hybridization signal from the target specific probe with a “control” probe (e.g. a probe for a “housekeeping gene) provides an estimate of the relative expression level of the target nucleic acid.

Alternatively, the target factor mRNA can be directly quantified in a Northern blot. In brief, the mRNA is isolated from a given cell sample using, for example, an acid guanidinium-phenol-chloroform extraction method. The mRNA is then electrophoresed to separate the mRNA species and the mRNA is transferred from the gel to a nitrocellulose membrane. As with the Southern blots, labeled probes are used to identify and/or quantify the target mRNA. Appropriate controls (e.g. probes to housekeeping genes) provide a reference for evaluating relative expression level.

An alternative means for determining the Tsp42Ee, and/or Tsp42El, and/or sgg expression level is in situ hybridization. In situ hybridization assays are well known (e.g., Angerer (1987) Meth. Enzymol 152: 649). Generally, in situ hybridization comprises the following major steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application.

In some applications it is necessary to block the hybridization capacity of repetitive sequences. Thus, in some embodiments, tRNA, human genomic DNA, or Cot-1 DNA is used to block non-specific hybridization.

3) Amplification-Based Assays.

In another embodiment, amplification-based assays can be used to measure Tsp42Ee, and/or Tsp42El, and/or sgg factor expression (transcription) level. In such amplification-based assays, the target nucleic acid sequences (i.e., Tsp42Ee, and/or Tsp42El, and/or sgg nucleic acid(s)) act as template(s) in amplification reaction(s) (e.g. Polymerase Chain Reaction (PCR) or reverse-transcription PCR (RT-PCR)). In a quantitative amplification, the amount of amplification product will be proportional to the amount of template in the original sample. Comparison to appropriate (e.g. healthy tissue or cells unexposed to the test agent) controls provides a measure of the Tsp42Ee, and/or Tsp42El, and/or sgg transcript level.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis et al. (1990) PCR Protocols, A Guide to Methods and Applications, Academic Press, Inc. N.Y.). One approach, for example, involves simultaneously co-amplifying a known quantity of a control sequence using the same primers as those used to amplify the target. This provides an internal standard that may be used to calibrate the PCR reaction.

One preferred internal standard is a synthetic AW106 cRNA. The AW106 cRNA is combined with RNA isolated from the sample according to standard techniques known to those of skill in the art. The RNA is then reverse transcribed using a reverse transcriptase to provide copy DNA. The cDNA sequences are then amplified (e.g., by PCR) using labeled primers. The amplification products are separated, typically by electrophoresis, and the amount of labeled nucleic acid (proportional to the amount of amplified product) is determined. The amount of mRNA in the sample is then calculated by comparison with the signal produced by the known AW106 RNA standard. Detailed protocols for quantitative PCR are provided in PCR Protocols, A Guide to Methods and Applications, Innis et al. (1990) Academic Press, Inc. N.Y. The known nucleic acid sequence(s) for Tsp42Ee, and/or Tsp42El, and/or sgg are sufficient to enable one of skill to routinely select primers to amplify any portion of the gene.

4) Hybridization Formats and Optimization of Hybridization

a) Array-Based Hybridization Formats.

In one embodiment, the methods of this invention can be utilized in array-based hybridization formats. Arrays are a multiplicity of different “probe” or “target” nucleic acids (or other compounds) attached to one or more surfaces (e.g., solid, membrane, or gel). In a preferred embodiment, the multiplicity of nucleic acids (or other moieties) is attached to a single contiguous surface or to a multiplicity of surfaces juxtaposed to each other.

In an array format a large number of different hybridization reactions can be run essentially “in parallel.” This provides rapid, essentially simultaneous, evaluation of a number of hybridizations in a single “experiment”. Methods of performing hybridization reactions in array based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211).

Arrays, particularly nucleic acid arrays can be produced according to a wide variety of methods well known to those of skill in the art. For example, in a simple embodiment, “low density” arrays can simply be produced by spotting (e.g. by hand using a pipette) different nucleic acids at different locations on a solid support (e.g. a glass surface, a membrane, etc.).

This simple spotting, approach has been automated to produce high density spotted arrays (see, e.g., U.S. Pat. No. 5,807,522). This patent describes the use of an automated system that taps a microcapillary against a surface to deposit a small volume of a biological sample. The process is repeated to generate high density arrays.

Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high density oligonucleotide arrays. Synthesis of high density arrays is also described in U.S. Pat. Nos. 5,744,305, 5,800,992 and 5,445,934.

b) Other Hybridization Formats.

As indicated above a variety of nucleic acid hybridization formats are known to those skilled in the art. For example, common formats include sandwich assays and competition or displacement assays. Such assay formats are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587.

Sandwich assays are commercially useful hybridization assays for detecting or isolating nucleic acid sequences. Such assays utilize a “capture” nucleic acid covalently immobilized to a solid support and a labeled “signal” nucleic acid in solution. The sample will provide the target nucleic acid. The “capture” nucleic acid and “signal” nucleic acid probe hybridize with the target nucleic acid to form a “sandwich” hybridization complex. To be most effective, the signal nucleic acid should not hybridize with the capture nucleic acid.

Typically, labeled signal nucleic acids are used to detect hybridization. Complementary nucleic acids or signal nucleic acids may be labeled by any one of several methods typically used to detect the presence of hybridized polynucleotides. The most common method of detection is the use of autoradiography with ³H, ¹²⁵I, ³⁵S, ¹⁴C, or ³²P-labelled probes or the like. Other labels include ligands that bind to labeled antibodies, fluorophores, chemi-luminescent agents, enzymes, and antibodies which can serve as specific binding pair members for a labeled ligand.

Detection of a hybridization complex may require the binding of a signal generating complex to a duplex of target and probe polynucleotides or nucleic acids. Typically, such binding occurs through ligand and anti-ligand interactions as between a ligand-conjugated probe and an anti-ligand conjugated with a signal.

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario) and Q Beta Replicase systems.

c) Optimization of Hybridization Conditions.

Nucleic acid hybridization simply involves providing a denatured probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids, or in the addition of chemical agents, or the raising of the pH. Under low stringency conditions (e.g., low temperature and/or high salt and/or high target concentration) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

One of skill in the art will appreciate that hybridization conditions may be selected to provide any degree of stringency. In a preferred embodiment, hybridization is performed at low stringency to ensure hybridization and then subsequent washes are performed at higher stringency to eliminate mismatched hybrid duplexes. Successive washes may be performed at increasingly higher stringency (e.g., down to as low as 0.25×SSPE at 37° C. to 70° C.) until a desired level of hybridization specificity is obtained. Stringency can also be increased by addition of agents such as formamide. Hybridization specificity may be evaluated by comparison of hybridization to the test probes with hybridization to the various controls that can be present.

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular probes of interest.

In a preferred embodiment, background signal is reduced by the use of a blocking reagent (e.g., tRNA, sperm DNA, cot-1 DNA, etc.) during the hybridization to reduce non-specific binding. The use of blocking agents in hybridization is well known to those of skill in the art (see, e.g., Chapter 8 in P. Tijssen, supra.)

Methods of optimizing hybridization conditions are well known to those of skill in the art (see, e.g., Tijssen (1993) Laboratory Techniques in Biochemistry and Molecular Biology, Vol. 24: Hybridization With Nucleic Acid Probes, Elsevier, N.Y.).

Optimal conditions are also a function of the sensitivity of label (e.g., fluorescence) detection for different combinations of substrate type, fluorochrome, excitation and emission bands, spot size and the like. Low fluorescence background surfaces can be used (see, e.g., Chu (1992) Electrophoresis 13:105-114). The sensitivity for detection of spots (“target elements”) of various diameters on the candidate surfaces can be readily determined by, e.g., spotting a dilution series of fluorescently end labeled DNA fragments. These spots are then imaged using conventional fluorescence microscopy. The sensitivity, linearity, and dynamic range achievable from the various combinations of fluorochrome and solid surfaces (e.g., glass, fused silica, etc.) can thus be determined. Serial dilutions of pairs of fluorochrome in known relative proportions can also be analyzed. This determines the accuracy with which fluorescence ratio measurements reflect actual fluorochrome ratios over the dynamic range permitted by the detectors and fluorescence of the substrate upon which the probe has been fixed.

d) Labeling and Detection of Nucleic Acids.

The probes used herein for detection of Tsp42Ee, and/or Tsp42El, and/or sgg factor expression levels can be full length or less than the full length of the Tsp42Ee, and/or Tsp42El, and/or sgg mRNA(s). Shorter probes are empirically tested for specificity. Preferred probes are sufficiently long so as to specifically hybridize with the target nucleic acid(s) under stringent conditions. The preferred size range is from about 20 bases to the length of the n Tsp42Ee, and/or Tsp42El, and/or sgg mRNA, more preferably from about 30 bases to the length of the Tsp42Ee, and/or Tsp42El, and/or sgg mRNA, and most preferably from about 40 bases to the length of the Tsp42Ee, and/or Tsp42El, and/or sgg mRNA.

The probes are typically labeled, with a detectable label. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like, see, e.g., Molecular Probes, Eugene, Oreg., USA), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

A fluorescent label is preferred because it provides a very strong signal with low background. It is also optically detectable at high resolution and sensitivity through a quick scanning procedure. The nucleic acid samples can all be labeled with a single label, e.g., a single fluorescent label. Alternatively, in another embodiment, different nucleic acid samples can be simultaneously hybridized where each nucleic acid sample has a different label. For instance, one target could have a green fluorescent label and a second target could have a red fluorescent label. The scanning step will distinguish sites of binding of the red label from those binding the green fluorescent label. Each nucleic acid sample (target nucleic acid) can be analyzed independently from one another.

Suitable chromogens which can be employed include those molecules and compounds which absorb light in a distinctive range of wavelengths so that a color can be observed or, alternatively, which emit light when irradiated with radiation of a particular wave length or wave length range, e.g., fluorescers.

Desirably, fluorescent labels should absorb light above about 300 nm, preferably about 350 nm, and more preferably above about 400 nm, usually emitting at wavelengths greater than about 10 nm higher than the wavelength of the light absorbed. It should be noted that the absorption and emission characteristics of the bound dye can differ from the unbound dye. Therefore, when referring to the various wavelength ranges and characteristics of the dyes, it is intended to indicate the dyes as employed and not the dye which is unconjugated and characterized in an arbitrary solvent.

Detectable signal can also be provided by chemiluminescent and bioluminescent sources. Chemiluminescent sources include a compound which becomes electronically excited by a chemical reaction and can then emit light which serves as the detectable signal or donates energy to a fluorescent acceptor. Alternatively, luciferins can be used in conjunction with luciferase or lucigenins to provide bioluminescence.

Spin labels are provided by reporter molecules with an unpaired electron spin which can be detected by electron spin resonance (ESR) spectroscopy. Exemplary spin labels include organic free radicals, transitional metal complexes, particularly vanadium, copper, iron, and manganese, and the like. Exemplary spin labels include nitroxide free radicals.

The label may be added to the target (sample) nucleic acid(s) prior to, or after the hybridization. So called “direct labels” are detectable labels that are directly attached to or incorporated into the target (sample) nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization With Nucleic Acid Probes, P. Tijssen, ed. Elsevier, N.Y., (1993)).

Fluorescent labels are easily added during an in vitro transcription reaction. Thus, for example, fluorescein labeled UTP and CTP can be incorporated into the RNA produced in an in vitro transcription.

The labels can be attached directly or through a linker moiety. In general, the site of label or linker-label attachment is not limited to any specific position. For example, a label may be attached to a nucleoside, nucleotide, or analogue thereof at any position that does not interfere with detection or hybridization as desired. For example, certain Label-ON Reagents from Clontech (Palo Alto, Calif.) provide for labeling interspersed throughout the phosphate backbone of an oligonucleotide and for terminal labeling at the 3′ and 5′ ends. As shown for example herein, labels can be attached at positions on the ribose ring or the ribose can be modified and even eliminated as desired. The base moieties of useful labeling reagents can include those that are naturally occurring or modified in a manner that does not interfere with the purpose to which they are put. Modified bases include but are not limited to 7-deaza A and G, 7-deaza-8-aza A and G, and other heterocyclic moieties.

It will be recognized that fluorescent labels are not to be limited to single species organic molecules, but include inorganic molecules, multi-molecular mixtures of organic and/or inorganic molecules, crystals, heteropolymers, and the like. Thus, for example, CdSe—CdS core-shell nanocrystals enclosed in a silica shell can be easily derivatized for coupling to a biological molecule (Bruchez et al. (1998) Science, 281: 2013-2016). Similarly, highly fluorescent quantum dots (zinc sulfide-capped cadmium selenide) have been covalently coupled to biomolecules for use in ultrasensitive biological detection (Warren and Nie (1998) Science, 281: 2016-2018).

B) Polypeptide-Based Assays.

1) Assay Formats.

In addition to, or in alternative to, the detection of Tsp42Ee, and/or Tsp42El, and/or sgg nucleic acid expression level(s), alterations in expression of Tsp42Ee, and/or Tsp42El, and/or sgg can be detected and/or quantified by detecting and/or quantifying the amount and/or activity of translated Tsp42Ee, and/or Tsp42El, and/or sgg polypeptide(s).

2) Detection of Expressed Protein

The Tsp42Ee, and/or Tsp42El, and/or sgg polypeptide(s) can be detected and quantified by any of a number of methods well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, western blotting, and the like.

In one preferred embodiment, the Tsp42Ee, and/or Tsp42El, and/or sgg polypeptide(s) are detected/quantified in an electrophoretic protein separation (e.g. a 1- or 2-dimensional electrophoresis). Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

In another preferred embodiment, Western blot (immunoblot) analysis is used to detect and quantify the presence of polypeptide(s) of this invention in the sample. This technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the target polypeptide(s).

The antibodies specifically bind to the target polypeptide(s) and may be directly labeled or alternatively may be subsequently detected using labeled antibodies (e.g., labeled sheep anti-mouse antibodies) that specifically bind to the a domain of the antibody.

In preferred embodiments, the Tsp42Ee, and/or Tsp42El, and/or sgg polypeptide(s) are detected using an immunoassay. As used herein, an immunoassay is an assay that utilizes an antibody to specifically bind to the analyte (e.g., the target polypeptide(s)). The immunoassay is thus characterized by detection of specific binding of a polypeptide of this invention to an antibody as opposed to the use of other physical or chemical properties to isolate, target, and quantify the analyte.

Any of a number of well recognized immunological binding assays (see, e.g., U.S. Pat. Nos. 4,366,241; 4,376,110; 4,517,288; and 4,837,168) are well suited to detection or quantification of the polypeptide(s) identified herein. For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

Immunological binding assays (or immunoassays) typically utilize a “capture agent” to specifically bind to and often immobilize the analyte (Tsp42Ee, and/or Tsp42El, and/or sgg). In preferred embodiments, the capture agent is an antibody.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled polypeptide or a labeled antibody that specifically recognizes the already bound target polypeptide. Alternatively, the labeling agent may be a third moiety, such as another antibody, that specifically binds to the capture agent/polypeptide complex.

Other proteins capable of specifically binding immunoglobulin constant regions, such as protein A or protein G may also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

Preferred immunoassays for detecting the target polypeptide(s) are either competitive or noncompetitive. Noncompetitive immunoassays are assays in which the amount of captured analyte is directly measured. In one preferred “sandwich” assay, for example, the capture agents (antibodies) can be bound directly to a solid substrate where they are immobilized. These immobilized antibodies then capture the target polypeptide present in the test sample. The target polypeptide thus immobilized is then bound by a labeling agent, such as a second antibody bearing a label.

In competitive assays, the amount of analyte (Tsp42Ee, and/or Tsp42El, and/or sgg protein) present in the sample is measured indirectly by measuring the amount of an added (exogenous) analyte displaced (or competed away) from a capture agent (antibody) by the analyte present in the sample. In one competitive assay, a known amount of, in this case, labeled polypeptide is added to the sample and the sample is then contacted with a capture agent. The amount of labeled polypeptide bound to the antibody is inversely proportional to the concentration of target polypeptide present in the sample.

In one particularly preferred embodiment, the antibody is immobilized on a solid substrate. The amount of target polypeptide bound to the antibody may be determined either by measuring the amount of target polypeptide present in an polypeptide/antibody complex, or alternatively by measuring the amount of remaining uncomplexed polypeptide.

The immunoassay methods of the present invention include an enzyme immunoassay (EIA) which utilizes, depending on the particular protocol employed, unlabeled or labeled (e.g., enzyme-labeled) derivatives of polyclonal or monoclonal antibodies or antibody fragments or single-chain antibodies that bind Tsp42Ee, and/or Tsp42El, and/or sgg, either alone or in combination. In the case where the antibody that binds the Tsp42Ee, and/or Tsp42El, and/or sgg polypeptide(s) is not labeled, a different detectable marker, for example, an enzyme-labeled antibody capable of binding to the monoclonal antibody which binds the Tsp42Ee, and/or Tsp42El, and/or sgg polypeptide, can be employed. Any of the known modifications of EIA, for example, enzyme-linked immunoabsorbent assay (ELISA), may also be employed. As indicated above, also contemplated by the present invention are immunoblotting immunoassay techniques such as western blotting employing an enzymatic detection system.

The immunoassay methods of the present invention can also include other known immunoassay methods, for example, fluorescent immunoassays using antibody conjugates or antigen conjugates of fluorescent substances such as fluorescein or rhodamine, latex agglutination with antibody-coated or antigen-coated latex particles, haemagglutination with antibody-coated or antigen-coated red blood corpuscles, and immunoassays employing an avidin-biotin or strepavidin-biotin detection systems, and the like.

The particular parameters employed in the immunoassays of the present invention can vary widely depending on various factors such as the concentration of antigen in the sample, the nature of the sample, the type of immunoassay employed and the like. Optimal conditions can be readily established by those of ordinary skill in the art. In certain embodiments, the amount of antibody that binds the Tsp42Ee, and/or Tsp42El, and/or sgg polypeptide is typically selected to give 50% binding of detectable marker in the absence of sample. If purified antibody is used as the antibody source, the amount of antibody used per assay will generally range from about 1 ng to about 100 ng. Typical assay conditions include a temperature range of about 4° C. to about 45° C., preferably about 25° C. to about 37° C., and most preferably about 25° C., a pH value range of about 5 to 9, preferably about 7, and an ionic strength varying from that of distilled water to that of about 0.2M sodium chloride, preferably about that of 0.15M sodium chloride. Times will vary widely depending upon the nature of the assay, and generally range from about 0.1 minute to about 24 hours. A wide variety of buffers, for example PBS, may be employed, and other reagents such as salt to enhance ionic strength, proteins such as serum albumins, stabilizers, biocides and non-ionic detergents can also be included.

The assays of this invention are scored (as positive or negative or quantity of target polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of target polypeptide concentration.

Antibodies for use in the various immunoassays described herein, are commercially available or can be produced using standard methods well know to those of skill in the art.

It will also be recognized that antibodies can be prepared by any of a number of commercial services (e.g., Berkeley antibody laboratories, Bethyl Laboratories, Anawa, Eurogenetec, etc.).

C) Assay Optimization.

The assays of this invention have immediate utility in screening for agents that modulate the expression or activity of Tsp42Ee, and/or Tsp42El, and/or sgg by a cell, tissue or organism. The assays of this invention can be optimized for use in particular contexts, depending, for example, on the source and/or nature of the biological sample and/or the particular test agents, and/or the analytic facilities available. Thus, for example, optimization can involve determining optimal conditions for binding assays, optimum sample processing conditions (e.g. preferred PCR conditions), hybridization conditions that maximize signal to noise, protocols that improve throughput, etc. In addition, assay formats can be selected and/or optimized according to the availability of equipment and/or reagents. Thus, for example, where commercial antibodies or ELISA kits are available it may be desired to assay protein concentration. Conversely, where it is desired to screen for modulators that alter transcription the Tsp42Ee, and/or Tsp42El, and/or sgg gene(s), nucleic acid based assays are preferred.

Routine selection and optimization of assay formats is well known to those of ordinary skill in the art.

II. Pre-Screening for Agents that Bind a Tsp42Ee, and/or Tsp42El, and/or sgg Gene(s) or Gene Products.

In certain embodiments it is desired to pre-screen test agents for the ability to interact with (e.g. specifically bind to) a Tsp42Ee, and/or Tsp42El, and/or sgg polypeptide or to a nucleic acid encoding such a polypeptide. Specifically binding test agents are more likely to interact with and thereby modulate Tsp42Ee, and/or Tsp42El, and/or sgg expression and/or activity. Thus, in some preferred embodiments, the test agent(s) are pre-screened for binding to Tsp42Ee, and/or Tsp42El, and/or sgg nucleic acids or to Tsp42Ee, and/or Tsp42El, and/or sgg proteins before performing the more complex assays described above.

In one embodiment, such pre-screening is accomplished with simple binding assays. Means of assaying for specific binding or the binding affinity of a particular ligand for a nucleic acid or for a protein are well known to those of skill in the art. In preferred binding assays, the Tsp42Ee, and/or Tsp42El, and/or sgg protein or nucleic acid is immobilized and exposed to a test agent (which can be labeled), or alternatively, the test agent(s) are immobilized and exposed to an Tsp42Ee, and/or Tsp42El, and/or sgg protein or to a Tsp42Ee, and/or Tsp42El, and/or sgg nucleic acid (which can be labeled). The immobilized moiety is then washed to remove any unbound material and the bound test agent or bound Tsp42Ee, and/or Tsp42El, and/or sgg nucleic acid or protein is detected (e.g. by detection of a label attached to the bound molecule). The amount of immobilized label is proportional to the degree of binding between the Tsp42Ee, and/or Tsp42El, and/or sgg protein or nucleic acid and the test agent.

III. Scoring the Assays.

As indicated above, methods of screening for modulators of Tsp42Ee, and/or Tsp42El, and/or sgg expression typically involve contacting a cell, tissue, organism, animal with one or more test agents and evaluating changes in Tsp42Ee, and/or Tsp42El, and/or sgg nucleic acid transcription and/or translation or Tsp42Ee, and/or Tsp42El, and/or sgg factor protein expression or activity. To screen for potential modulators, the assays described above are performed in the after administering and/or in the presence of one or more test agents using biological samples from cells and/or tissues and/or organs and/or organisms exposed to one or more test agents. The Tsp42Ee, and/or Tsp42El, and/or sgg activity and/or expression level is determined and, in a preferred embodiment, compared to the activity level(s) observed in “control” assays (e.g., the same assays lacking the test agent). A difference between the Tsp42Ee, and/or Tsp42El, and/or sgg expression and/or activity in the “test” assay as compared to the control assay indicates that the test agent is a “modulator” of Tsp42Ee, and/or Tsp42El, and/or sgg expression and/or activity.

In a preferred embodiment, the assays of this invention level are deemed to show a positive result, e.g. elevated expression and/or activity of Tsp42Ee, and/or Tsp42El, and/or sgg, when the measured protein or nucleic acid level or protein activity is greater than the level measured or known for a control sample (e.g. either a level known or measured for a normal healthy cell, tissue or organism mammal of the same species not exposed to the or putative modulator (test agent), or a “baseline/reference” level determined at a different tissue and/or a different time for the same individual). In a particularly preferred embodiment, the assay is deemed to show a positive result when the difference between sample and “control” is statistically significant (e.g. at the 85% or greater, preferably at the 90% or greater, more preferably at the 95% or greater and most preferably at the 98% or greater confidence level).

IV. High Throughput Screening.

The assays of this invention are also amenable to “high-throughput” modalities. Conventionally, new chemical entities with useful properties (e.g., modulation of Tsp42Ee, and/or Tsp42El, and/or sgg expression or activity) are generated by identifying a chemical compound (called a “lead compound”) with some desirable property or activity, creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

In one preferred embodiment, high throughput screening methods involve providing a library containing a large number of compounds (candidate compounds) potentially having the desired activity. Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A) Combinatorial Chemical Libraries

Recently, attention has focused on the use of combinatorial chemical libraries to assist in the generation of new chemical compound leads. A combinatorial chemical library is a collection of diverse chemical compounds generated by either chemical synthesis or biological synthesis by combining a number of chemical “building blocks” such as reagents. For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, one commentator has observed that the systematic, combinatorial mixing of 100 interchangeable chemical building blocks results in the theoretical synthesis of 100 million tetrameric compounds or 10 billion pentameric compounds (Gallop et al. (1994) 37(9): 1233-1250).

Preparation and screening of combinatorial chemical libraries is well known to those of skill in the art. Such combinatorial chemical libraries include, but are not limited to, peptide libraries (see, e.g., U.S. Pat. No. 5,010,175, Furka (1991) Int. J. Pept. Prot. Res., 37: 487-493, Houghton et al. (1991) Nature, 354: 84-88). Peptide synthesis is by no means the only approach envisioned and intended for use with the present invention. Other chemistries for generating chemical diversity libraries can also be used. Such chemistries include, but are not limited to: peptoids (PCT Publication No WO 91/19735, 26 Dec. 1991), encoded peptides (PCT Publication WO 93/20242, 14 Oct. 1993), random bio-oligomers (PCT Publication WO 92/00091, 9 Jan. 1992), benzodiazepines (U.S. Pat. No. 5,288,514), diversomers such as hydantoins, benzodiazepines and dipeptides (Hobbs et al., (1993) Proc. Nat. Acad. Sci. USA 90: 6909-6913), vinylogous polypeptides (Hagihara et al. (1992) J. Amer. Chem. Soc. 114: 6568), nonpeptidal peptidomimetics with a Beta-D-Glucose scaffolding (Hirschmann et al., (1992) J. Amer. Chem. Soc. 114: 9217-9218), analogous organic syntheses of small compound libraries (Chen et al. (1994) J. Amer. Chem. Soc. 116: 2661), oligocarbamates (Cho, et al., (1993) Science 261:1303), and/or peptidyl phosphonates (Campbell et al., (1994) J. Org. Chem. 59: 658). See, generally, Gordon et al., (1994) J. Med. Chem. 37:1385, nucleic acid libraries (see, e.g., Strategene, Corp.), peptide nucleic acid libraries (see, e.g., U.S. Pat. No. 5,539,083) antibody libraries (see, e.g., Vaughn et al. (1996) Nature Biotechnology, 14(3): 309-314), and PCT/US96/10287), carbohydrate libraries (see, e.g., Liang et al. (1996) Science, 274: 1520-1522, and U.S. Pat. No. 5,593,853), and small organic molecule libraries (see, e.g., benzodiazepines, Baum (1993) C&EN, January 18, page 33, isoprenoids U.S. Pat. No. 5,569,588, thiazolidinones and metathiazanones U.S. Pat. No. 5,549,974, pyrrolidines U.S. Pat. Nos. 5,525,735 and 5,519,134, morpholino compounds U.S. Pat. No. 5,506,337, benzodiazepines 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

B) High Throughput Assays of Chemical Libraries.

Any of the assays for agents that modulate expression of Tsp42Ee, and/or Tsp42El, and/or sgg or that alter the binding specificity and/or activity of Tsp42Ee, and/or Tsp42El, and/or sgg polypeptides are amenable to high throughput screening. As described above, having determined that Tsp42Ee, and/or Tsp42El, and/or sgg are associated with an organism's behavioral response to consumption of alcohol or other substances of abuse, it is believe that modulators can have significant therapeutic value. Certain preferred assays detect increases of transcription (i.e., increases of mRNA production) by the test compound(s), increases of protein expression by the test compound(s), or binding to the gene (e.g., gDNA, or cDNA) or gene product (e.g., mRNA or expressed protein) by the test compound(s). Alternatively, the assay can detect inhibition of the characteristic activity of the Tsp42Ee, and/or Tsp42El, and/or sgg.

High throughput assays for the presence, absence, or quantification of particular nucleic acids or protein products are well known to those of skill in the art. Similarly, binding assays are similarly well known. Thus, for example, U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols the various high throughput. Thus, for example, Zymark Corp. provides technical bulletins describing screening systems for detecting the modulation of gene transcription, ligand binding, and the like.

V. Kits.

In still another embodiment, this invention provides kits for practice of the assays or use of the compositions described herein. In one preferred embodiment, the kits comprise one or more containers containing antibodies and/or nucleic acid probes and/or substrates suitable for detection of Tsp42Ee, and/or Tsp42El, and/or sgg expression and/or activity levels. The kits can optionally include any reagents and/or apparatus to facilitate practice of the assays described herein. Such reagents include, but are not limited to buffers, labels, labeled antibodies, labeled nucleic acids, filter sets for visualization of fluorescent labels, blotting membranes, and the like.

In another embodiment, the kits can comprise a container containing a Tsp42Ee, and/or Tsp42El, and/or sgg protein(s), and/or a vector encoding a Tsp42Ee, and/or a Tsp42El, and/or an sgg, and/or a cell comprising such a vector.

In addition, the kits can, optionally, include instructional materials containing directions (i.e., protocols) for the practice of the assay methods of this invention or the administration of the compositions described here along with counterindications. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

VI. Modulator Databases.

In certain embodiments, the agents that score positively in the assays described herein (e.g. show an ability to inhibit and/or to increase the expression or activity of a Tsp42Ee, and/or a Tsp42El, and/or an sgg) can be entered into a database of putative modulators of an organism's response to consumption of alcohol or other substances of abuse. The term database refers to a means for recording and retrieving information. In certain embodiments the database also provides means for sorting and/or searching the stored information. The database can comprise any convenient media including, but not limited to, paper systems, card systems, mechanical systems, electronic systems, optical systems, magnetic systems or combinations thereof. Typical databases include electronic (e.g. computer-based) databases. Computer systems for use in storage and manipulation of databases are well known to those of skill in the art and include, but are not limited to “personal computer systems”, mainframe systems, distributed nodes on an inter- or intra-net, data or databases stored in specialized hardware (e.g. in microchips), and the like.

VII. Altering a Tsp42Ee, a Tsp42El and/or a Glycogen Synthase Kinase-3 expression and/or activity.

Tsp42Ee, Tsp42 μl and/or a Glycogen Synthase Kinase-3 (GSK-3) expression can upregulated or inhibited using a wide variety of approaches known to those of skill in the art. For example, methods of inhibiting expression include, but are not limited to antisense molecules, target-specific ribozymes, target-specific catalytic DNAs, intrabodies directed against target proteins, RNAi, gene therapy approaches that knock out Tsp42Ee, a Tsp42El and/or a Glycogen Synthase Kinase-3, and small organic molecules that inhibit expression of the target gene(s)/

Tsp42Ee, a Tsp42El and/or a GSK-3 expression and/or activity can be up-regulated by introducing constructs expressing Tsp42Ee, a Tsp42El and/or a GSK-3 into the cell (e.g. using gene therapy approaches) or upregulating endogenous expression of Tsp42Ee, a Tsp42El and/or a GSK-3 (e.g. using agents identified in the screening assays of this invention).

A) Antisense Approaches.

Tsp42Ee, a Tsp42El and/or a GSK-3 gene expression can be down-regulated or entirely inhibited by the use of antisense molecules. An “antisense sequence or antisense nucleic acid” is a nucleic acid that is complementary to the coding Tsp42Ee, a Tsp42El and/or a GSK-3 mRNA nucleic acid sequence or a subsequence thereof. Binding of the antisense molecule to the Tsp42Ee, a Tsp42El and/or a GSK-3 mRNA interferes with normal translation of the Tsp42Ee, a Tsp42El and/or a GSK-3 polypeptide.

Thus, in accordance with preferred embodiments of this invention, preferred antisense molecules include oligonucleotides and oligonucleotide analogs that are hybridizable with Tsp42Ee, a Tsp42El and/or a GSK-3 messenger RNA. This relationship is commonly denominated as “antisense.” The oligonucleotides and oligonucleotide analogs are able to inhibit the function of the RNA, either its translation into protein, its translocation into the cytoplasm, or any other activity necessary to its overall biological function. The failure of the messenger RNA to perform all or part of its function results in a reduction or complete inhibition of expression of Tsp42Ee, a Tsp42El and/or a GSK-3 polypeptides.

In the context of this invention, the term “oligonucleotide” refers to a polynucleotide formed from naturally-occurring bases and/or cyclofuranosyl groups joined by native phosphodiester bonds. This term effectively refers to naturally-occurring species or synthetic species formed from naturally-occurring subunits or their close homologs. The term “oligonucleotide” may also refer to moieties which function similarly to oligonucleotides, but which have non naturally-occurring portions. Thus, oligonucleotides may have altered sugar moieties or inter-sugar linkages. Exemplary among these are the phosphorothioate and other sulfur containing species which are known for use in the art. In accordance with some preferred embodiments, at least one of the phosphodiester bonds of the oligonucleotide has been substituted with a structure which functions to enhance the ability of the compositions to penetrate into the region of cells where the RNA whose activity is to be modulated is located. It is preferred that such substitutions comprise phosphorothioate bonds, methyl phosphonate bonds, or short chain alkyl or cycloalkyl structures. In accordance with other preferred embodiments, the phosphodiester bonds are substituted with structures which are, at once, substantially non-ionic and non-chiral, or with structures which are chiral and enantiomerically specific. Persons of ordinary skill in the art will be able to select other linkages for use in the practice of the invention.

In one particularly preferred embodiment, the internucleotide phosphodiester linkage is replaced with a peptide linkage. Such peptide nucleic acids tend to show improved stability, penetrate the cell more easily, and show enhances affinity for their target. Methods of making peptide nucleic acids are known to those of skill in the art (see, e.g., U.S. Pat. Nos. 6,015,887, 6,015,710, 5,986,053, 5,977,296, 5,902,786, 5,864,010, 5,786,461, 5,773,571, 5,766,855, 5,736,336, 5,719,262, and 5,714,331).

Oligonucleotides may also include species which include at least some modified base forms. Thus, purines and pyrimidines other than those normally found in nature may be so employed. Similarly, modifications on the furanosyl portions of the nucleotide subunits may also be effected, as long as the essential tenets of this invention are adhered to. Examples of such modifications are 2′-O-alkyl- and 2′-halogen-substituted nucleotides. Some specific examples of modifications at the 2′ position of sugar moieties which are useful in the present invention are OH, SH, SCH₃, F, OCH₃, OCN, O(CH₂)[n]NH₂ or O(CH₂)[n]CH₃, where n is from 1 to about 10, and other substituents having similar properties.

Such oligonucleotides are best described as being functionally interchangeable with natural oligonucleotides or synthesized oligonucleotides along natural lines, but which have one or more differences from natural structure. All such analogs are comprehended by this invention so long as they function effectively to hybridize with messenger RNA of Tsp42Ee, a Tsp42El and/or a GSK-3 to inhibit the function of that RNA.

The oligonucleotides in accordance with this invention preferably comprise from about 3 to about 50 subunits. It is more preferred that such oligonucleotides and analogs comprise from about 8 to about 25 subunits and still more preferred to have from about 12 to about 20 subunits. As will be appreciated, a subunit is a base and sugar combination suitably bound to adjacent subunits through phosphodiester or other bonds. The oligonucleotides used in accordance with this invention may be conveniently and routinely made through the well-known technique of solid phase synthesis. Equipment for such synthesis is sold by several vendors, including Applied Biosystems. Any other means for such synthesis may also be employed, however, the actual synthesis of the oligonucleotides is well within the talents of the routineer. It is also will known to prepare other oligonucleotide such as phosphorothioates and alkylated derivatives.

Using the known sequence of the Tsp42Ee, a Tsp42El and/or a GSK-3 gene/cDNA, appropriate and effective antisense oligonucleotide sequences can be readily determined.

B) Catalytic RNAs and DNAs

1) Ribozymes.

In another approach, Tsp42Ee, a Tsp42El and/or a GSK-3 expression can be inhibited by the use of ribozymes. As used herein, “ribozymes” are include RNA molecules that contain anti-sense sequences for specific recognition, and an RNA-cleaving enzymatic activity. The catalytic strand cleaves a specific site in a target (Tsp42Ee, a Tsp42El and/or a GSK-3) RNA, preferably at greater than stoichiometric concentration. Two “types” of ribozymes are particularly useful in this invention, the hammerhead ribozyme (Rossi et al. (1991) Pharmac. Ther. 50: 245-254) and the hairpin ribozyme (Hampel et al. (1990) Nucl. Acids Res. 18: 299-304, and U.S. Pat. No. 5,254,678).

Because both hammerhead and hairpin ribozymes are catalytic molecules having antisense and endoribonucleotidase activity, ribozyme technology has emerged as a potentially powerful extension of the antisense approach to gene inactivation. The ribozymes of the invention typically consist of RNA, but such ribozymes may also be composed of nucleic acid molecules comprising chimeric nucleic acid sequences (such as DNA/RNA sequences) and/or nucleic acid analogs (e.g., phosphorothioates).

Accordingly, within one aspect of the present invention ribozymes are provided which have the ability to inhibit Tsp42Ee, a Tsp42El and/or a GSK-3 expression. Such ribozymes may be in the form of a “hammerhead” (for example, as described by Forster and Symons (1987) Cell 48: 211-220,; Haseloff and Gerlach (1988) Nature 328: 596-600; Walbot and Bruening (1988) Nature 334: 196; Haseloff and Gerlach (1988) Nature 334: 585) or a “hairpin” (see, e.g. U.S. Pat. No. 5,254,678 and Hampel et al., European Patent Publication No. 0 360 257, published Mar. 26, 1990), and have the ability to specifically target, cleave and TSP42EE, A TSP42EL AND/OR A GSK-3 nucleic acids.

The sequence requirement for the hairpin ribozyme is any RNA sequence consisting of NNNBN*GUCNNNNNN (where N*G is the cleavage site, where B is any of G, C, or U, and where N is any of G, U, C, or A) (SEQ ID NO: 1). Suitable Tsp42Ee, a Tsp42El and/or a GSK-3 recognition or target sequences for hairpin ribozymes can be readily determined from the Tsp42Ee, a Tsp42El and/or a GSK-3 sequence.

The sequence requirement at the cleavage site for the hammerhead ribozyme is any RNA sequence consisting of NUX (where N is any of G, U, C, or A and X represents C, U, or A) can be targeted. Accordingly, the same target within the hairpin leader sequence, GUC, is useful for the hammerhead ribozyme. The additional nucleotides of the hammerhead ribozyme or hairpin ribozyme is determined by the target flanking nucleotides and the hammerhead consensus sequence (see Ruffner et al. (1990) Biochemistry 29: 10695-10702).

Cech et al. (U.S. Pat. No. 4,987,071,) has disclosed the preparation and use of certain synthetic ribozymes which have endoribonuclease activity. These ribozymes are based on the properties of the Tetrahymena ribosomal RNA self-splicing reaction and require an eight base pair target site. A temperature optimum of 50° C. is reported for the endoribonuclease activity. The fragments that arise from cleavage contain 5′ phosphate and 3′ hydroxyl groups and a free guanosine nucleotide added to the 5′ end of the cleaved RNA. The preferred ribozymes of this invention hybridize efficiently to target sequences at physiological temperatures, making them particularly well suited for use in vivo.

The ribozymes of this invention, as well as DNA encoding such ribozymes and other suitable nucleic acid molecules can be chemically synthesized using methods well known in the art for the synthesis of nucleic acid molecules. Alternatively, Promega, Madison, Wis., USA, provides a series of protocols suitable for the production of RNA molecules such as ribozymes. The ribozymes also can be prepared from a DNA molecule or other nucleic acid molecule (which, upon transcription, yields an RNA molecule) operably linked to an RNA polymerase promoter, e.g., the promoter for T7 RNA polymerase or SP6 RNA polymerase. Such a construct may be referred to as a vector. Accordingly, also provided by this invention are nucleic acid molecules, e.g., DNA or cDNA, coding for the ribozymes of this invention. When the vector also contains an RNA polymerase promoter operably linked to the DNA molecule, the ribozyme can be produced in vitro upon incubation with the RNA polymerase and appropriate nucleotides. In a separate embodiment, the DNA may be inserted into an expression cassette (see, e.g., Cotten and Birnstiel (1989) EMBO J 8(12):3861-3866; Hempel et al. (1989) Biochem. 28: 4929-4933, etc.).

After synthesis, the ribozyme can be modified by ligation to a DNA molecule having the ability to stabilize the ribozyme and make it resistant to RNase. Alternatively, the ribozyme can be modified to the phosphothio analog for use in liposome delivery systems. This modification also renders the ribozyme resistant to endonuclease activity.

The ribozyme molecule also can be in a host prokaryotic or eukaryotic cell in culture or in the cells of an organism/patient. Appropriate prokaryotic and eukaryotic cells can be transfected with an appropriate transfer vector containing the DNA molecule encoding a ribozyme of this invention. Alternatively, the ribozyme molecule, including nucleic acid molecules encoding the ribozyme, may be introduced into the host cell using traditional methods such as transformation using calcium phosphate precipitation (Dubensky et al. (1984) Proc. Natl. Acad. Sci., USA, 81: 7529-7533), direct microinjection of such nucleic acid molecules into intact target cells (Acsadi et al. (1991) Nature 352: 815-818), and electroporation whereby cells suspended in a conducting solution are subjected to an intense electric field in order to transiently polarize the membrane, allowing entry of the nucleic acid molecules. Other procedures include the use of nucleic acid molecules linked to an inactive adenovirus (Cotton et al. (1990) Proc. Natl. Acad. Sci., USA, 89 :6094), lipofection (Felgner et al. (1989) Proc. Natl. Acad. Sci. USA 84: 7413-7417), microprojectile bombardment (Williams et al. (1991) Proc. Natl. Acad. Sci., USA, 88: 2726-2730), polycation compounds such as polylysine, receptor specific ligands, liposomes entrapping the nucleic acid molecules, spheroplast fusion whereby E coli containing the nucleic acid molecules are stripped of their outer cell walls and fused to animal cells using polyethylene glycol, viral transduction, (Cline et al., (1985) Pharmac. Ther. 29: 69; and Friedmann et al. (1989) Science 244: 1275), and DNA ligand (Wu et al (1989) J. Biol. Chem. 264: 16985-16987), as well as psoralen inactivated viruses such as Sendai or Adenovirus. In one preferred embodiment, the ribozyme is introduced into the host cell utilizing a lipid, a liposome or a retroviral vector.

When the DNA molecule is operatively linked to a promoter for RNA transcription, the RNA can be produced in the host cell when the host cell is grown under suitable conditions favoring transcription of the DNA molecule. The vector can be, but is not limited to, a plasmid, a virus, a retrotransposon or a cosmid. Examples of such vectors are disclosed in U.S. Pat. No. 5,166,320. Other representative vectors include, but are not limited to adenoviral vectors (e.g., WO 94/26914, WO 93/9191; Kolls et al. (1994) PNAS 91(1):215-219; Kass-Eisler et al., (1993) Proc. Natl. Acad. Sci., USA, 90(24): 11498-502, Guzman et al. (1993) Circulation 88(6): 2838-48, 1993; Guzman et al. (1993) Cir. Res. 73(6):1202-1207, 1993; Zabner et al. (1993) Cell 75(2): 207-216; Li et al. (1993) Hum Gene Ther. 4(4): 403-409; Caillaud et al. (1993) Eur. JNeurosci. 5(10): 1287-1291), adeno-associated vector type 1 (“AAV-1”) or adeno-associated vector type 2 (“AAV-2”) (see WO 95/13365; Flotte et al. (1993) Proc. Natl. Acad. Sci., USA, 90(22):10613-10617), retroviral vectors (e.g., EP 0 415 731; WO 90/07936; WO 91/02805; WO 94/03622; WO 93/25698; WO 93/25234; U.S. Pat. No. 5,219,740; WO 93/11230; WO 93/10218) and herpes viral vectors (e.g., U.S. Pat. No. 5,288,641). Methods of utilizing such vectors in gene therapy are well known in the art, see, for example, Larrick and Burck (1991) Gene Therapy: Application of Molecular Biology, Elsevier Science Publishing Co., Inc., New York, N.Y., and Kreigler (1990) Gene Transfer and Expression: A Laboratory Manual, W.H. Freeman and Company, New York.

To produce ribozymes in vivo utilizing vectors, the nucleotide sequences coding for ribozymes are preferably placed under the control of a strong promoter such as the lac, SV40 late, SV40 early, or lambda promoters. Ribozymes are then produced directly from the transfer vector in vivo. Suitable transfector vectors for in vivo expression are discussed below.

2) Catalytic DNA

In a manner analogous to ribozymes, DNAs are also capable of demonstrating catalytic (e.g. nuclease) activity. While no such naturally-occurring DNAs are known, highly catalytic species have been developed by directed evolution and selection. Beginning with a population of 10¹⁴ DNAs containing 50 random nucleotides, successive rounds of selective amplification, enriched for individuals that best promote the Pb²⁺-dependent cleavage of a target ribonucleoside 3′-O—P bond embedded within an otherwise all-DNA sequence. By the fifth round, the population as a whole carried out this reaction at a rate of 0.2 min⁻¹. Based on the sequence of 20 individuals isolated from this population, a simplified version of the catalytic domain that operates in an intermolecular context with a turnover rate of 1 min⁻¹ (see, e.g., Breaker and Joyce (1994) Chem Biol 4: 223-229.

In later work, using a similar strategy, a DNA enzyme was made that could cleave almost any targeted RNA substrate under simulated physiological conditions. The enzyme is comprised of a catalytic domain of 15 deoxynucleotides, flanked by two substrate-recognition domains of seven to eight deoxynucleotides each. The RNA substrate is bound through Watson-Crick base pairing and is cleaved at a particular phosphodiester located between an unpaired purine and a paired pyrimidine residue. Despite its small size, the DNA enzyme has a catalytic efficiency (kcat/Km) of approximately 10⁹ M⁻¹ min⁻¹ under multiple turnover conditions, exceeding that of any other known nucleic acid enzyme. By changing the sequence of the substrate-recognition domains, the DNA enzyme can be made to target different RNA substrates (Santoro and Joyce (1997) Proc. Natl. Acad. Sci., USA, 94(9): 4262-4266). Modifying the appropriate targeting sequences (e.g. as described by Santoro and Joyce, supra.) the DNA enzyme can easily be retargeted to Tsp42Ee, a Tsp42El and/or a GSK-3 mRNA thereby acting like a ribozyme.

C) Knocking out TSP42EE, A TSP42EL AND/OR A GSK-3

In another approach, Tsp42Ee, a Tsp42El and/or a GSK-3 can be inhibited/downregulated simply by “knocking out” the gene. Typically this is accomplished by disrupting the Tsp42Ee, a Tsp42El and/or a GSK-3 gene, the promoter regulating the gene or sequences between the promoter and the gene. Such disruption can be specifically directed to Tsp42Ee, a Tsp42El and/or a GSK-3 by homologous recombination where a “knockout construct” contains flanking sequences complementary to the domain to which the construct is targeted. Insertion of the knockout construct (e.g. into the Tsp42Ee, a Tsp42El and/or a GSK-3 gene) results in disruption of that gene. The phrases “disruption of the gene” and “gene disruption” refer to insertion of a nucleic acid sequence into one region of the native DNA sequence (usually one or more exons) and/or the promoter region of a gene so as to decrease or prevent expression of that gene in the cell as compared to the wild-type or naturally occurring sequence of the gene. By way of example, a nucleic acid construct can be prepared containing a DNA sequence encoding an antibiotic resistance gene which is inserted into the DNA sequence that is complementary to the DNA sequence (promoter and/or coding region) to be disrupted. When this nucleic acid construct is then transfected into a cell, the construct will integrate into the genomic DNA. Thus, the cell and its progeny will no longer express the gene or will express it at a decreased level, as the DNA is now disrupted by the antibiotic resistance gene.

Knockout constructs can be produced by standard methods known to those of skill in the art. The knockout construct can be chemically synthesized or assembled, e.g., using recombinant DNA methods. The DNA sequence to be used in producing the knockout construct is digested with a particular restriction enzyme selected to cut at a location(s) such that a new DNA sequence encoding a marker gene can be inserted in the proper position within this DNA sequence. The proper position for marker gene insertion is that which will serve to prevent expression of the native gene; this position will depend on various factors such as the restriction sites in the sequence to be cut, and whether an exon sequence or a promoter sequence, or both is (are) to be interrupted (i.e., the precise location of insertion necessary to inhibit promoter function or to inhibit synthesis of the native exon). Preferably, the enzyme selected for cutting the DNA will generate a longer arm and a shorter arm, where the shorter arm is at least about 300 base pairs (bp). In some cases, it will be desirable to actually remove a portion or even all of one or more exons of the gene to be suppressed so as to keep the length of the knockout construct comparable to the original genomic sequence when the marker gene is inserted in the knockout construct. In these cases, the genomic DNA is cut with appropriate restriction endonucleases such that a fragment of the proper size can be removed.

The marker gene can be any nucleic acid sequence that is detectable and/or assayable, however typically it is an antibiotic resistance gene or other gene whose expression or presence in the genome can easily be detected. The marker gene is usually operably linked to its own promoter or to another strong promoter from any source that will be active or can easily be activated in the cell into which it is inserted; however, the marker gene need not have its own promoter attached as it may be transcribed using the promoter of the gene to be suppressed. In addition, the marker gene will normally have a polyA sequence attached to the 3′ end of the gene; this sequence serves to terminate transcription of the gene. Preferred marker genes are any antibiotic resistance gene including, but not limited to neo (the neomycin resistance gene) and beta-gal (beta-galactosidase).

After the genomic DNA sequence has been digested with the appropriate restriction enzymes, the marker gene sequence is ligated into the genomic DNA sequence using methods well known to the skilled artisan (see, e.g., Berger and Kimmel, Guide to Molecular Cloning Techniques, Methods in Enzymology volume 152 Academic Press, Inc., San Diego, Calif.; Sambrook et al. (1989) Molecular Cloning—A Laboratory Manual (2nd ed.) Vol. 1-3, Cold Spring Harbor Laboratory, Cold Spring Harbor Press, NY; and Current Protocols in Molecular Biology, F. M. Ausubel et al., eds., Current Protocols, a joint venture between Greene Publishing Associates, Inc. and John Wiley & Sons, Inc., (1994) Supplement). The ends of the DNA fragments to be ligated must be compatible; this is achieved by either cutting all fragments with enzymes that generate compatible ends, or by blunting the ends prior to ligation. Blunting is done using methods well known in the art, such as for example by the use of Klenow fragment (DNA polymerase I) to fill in sticky ends.

Suitable knockout constructs can be made and used to produce Tsp42Ee, a Tsp42El and/or a GSK-3 (homologue) knockout mice (see, e.g., Dorfman et al. (1996) Oncogene 13: 925-931). The knockout constructs can be delivered to cells in vivo using gene therapy delivery vehicles (e.g. retroviruses, liposomes, lipids, dendrimers, etc.) as described below. Methods of knocking out genes are well described in the literature and essentially routine to those of skill in the art (see, e.g., Thomas et al. (1986) Cell 44(3): 419-428; Thomas, et al. (1987) Cell 51(3): 503-512)1; Jasin and Berg (1988) Genes & Development 2: 1353-1363; Mansour, et al. (1988) Nature 336: 348-352; Brinster, et al. (1989) Proc Natl Acad Sci 86: 7087-7091; Capecchi (1989) Trends in Genetics 5(3): 70-76; Frohman and Martin (1989) Cell 56: 145-147; Hasty, et al. (1991) Mol Cell Bio 11(11): 5586-5591; Jeannotte, et al. (1991) Mol Cell Biol. 11(11): 557814 5585; and Mortensen, et al. (1992) Mol Cell Biol. 12(5): 2391-2395.

The use of homologous recombination to alter expression of endogenous genes is also described in detail in U.S. Pat. No. 5,272,071, WO 91/09955, WO 93/09222, WO 96/29411, WO 95/31560, and WO 91/12650.

D) Intrabodies.

In still another embodiment, Tsp42Ee, a Tsp42El and/or a GSK-3 expression/activity is inhibited by transfecting the subject cell(s) (e.g., cells of the vascular endothelium) with a nucleic acid construct that expresses an intrabody. An intrabody is an intracellular antibody, in this case, capable of recognizing and binding to a Tsp42Ee, a Tsp42El and/or a GSK-3 polypeptide. The intrabody is expressed by an “antibody cassette”, containing a sufficient number of nucleotides coding for the portion of an antibody capable of binding to the target (Tsp42Ee, a Tsp42El and/or a GSK-3 polypeptide) operably linked to a promoter that will permit expression of the antibody in the cell(s) of interest. The construct encoding the intrabody is delivered to the cell where the antibody is expressed intracellularly and binds to the target Tsp42Ee, a Tsp42El and/or a GSK-3, thereby disrupting the target from its normal action. This antibody is sometimes referred to as an “intrabody”.

In one preferred embodiment, the “intrabody gene” (antibody) of the antibody cassette would utilize a cDNA, encoding heavy chain variable (V_{H}) and light chain variable (V_{L}) domains of an antibody which can be connected at the DNA level by an appropriate oligonucleotide as a bridge of the two variable domains, which on translation, form a single peptide (referred to as a single chain variable fragment, “sFv”) capable of binding to a target such as an Tsp42Ee, a Tsp42El and/or a GSK-3 protein. The intrabody gene preferably does not encode an operable secretory sequence and thus the expressed antibody remains within the cell.

Anti-Tsp42Ee, -Tsp42El and/or -GSK-3 antibodies suitable for use/expression as intrabodies in the methods of this invention can be readily produced by a variety of methods. Such methods include, but are not limited to, traditional methods of raising “whole” polyclonal antibodies, which can be modified to form single chain antibodies, or screening of, e.g. phage display libraries to select for antibodies showing high specificity and/or avidity for Tsp42Ee, a Tsp42El and/or a GSK-3′. Such screening methods are described above in some detail.

The antibody cassette is delivered to the cell by any of the known means. This discloses the use of a fusion protein comprising a target moiety and a binding moiety. The target moiety brings the vector to the cell, while the binding moiety carries the antibody cassette. Other methods include, for example, Miller (1992) Nature 357: 455-460; Anderson (1992) Science 256: 808-813; Wu, et al. (1988) J. Biol. Chem. 263: 14621-14624. For example, a cassette containing these (anti-TSP42EE, A TSP42EL AND/OR A GSK-3) antibody genes, such as the sFv gene, can be targeted to a particular cell by a number of techniques including, but not limited to the use of tissue-specific promoters, the use of tissue specific vectors, and the like. Methods of making and using intrabodies are described in detail in U.S. Pat. No. 6,004,940.

E) Small Organic Molecules.

In still another embodiment, Tsp42Ee, a Tsp42El and/or a GSK-3 expression and/or Tsp42Ee, a Tsp42El and/or a GSK-3 protein activity can be inhibited by the use of small organic molecules. Such molecules include, but are not limited to molecules that specifically bind to the DNA comprising the Tsp42Ee, Tsp42El and/or GSK-3 promoter and/or coding region, molecules that bind to and complex with Tsp42Ee, Tsp42El and/or GSK-3 mRNA, molecules that inhibit the signaling pathway that results in Tsp42Ee, Tsp42El and/or GSK-3 upregulation, and molecules that bind to and/or compete with Tsp42Ee, Tsp42El and/or GSK-3 polypeptides. Small organic molecules effective at inhibiting Tsp42Ee, Tsp42El and/or GSK-3 expression can be identified with routine screening using the methods described herein.

The methods of inhibiting Tsp42Ee, Tsp42El and/or GSK-3 expression described above are meant to be illustrative and not limiting. In view of the teachings provided herein, other methods of inhibiting Tsp42Ee, Tsp42El and/or GSK-3 will be known to those of skill in the art.

F) Modes of Administration.

The mode of administration of the Tsp42Ee, Tsp42El and/or GSK-3 blocking or activating agent depends on the nature of the particular agent. Antisense molecules, catalytic RNAs (ribozymes), catalytic DNAs, small organic molecules, and other molecules (e.g. lipids, antibodies, etc.) used as Tsp42Ee, Tsp42El and/or GSK-3 inhibitors may be formulated as pharmaceuticals (e.g. with suitable excipient) and delivered using standard pharmaceutical formulation and delivery methods as described below. Antisense molecules, catalytic RNAs (ribozymes), catalytic DNAs, and additionally, knockout constructs, and constructs encoding intrabodies can be delivered and (if necessary) expressed in target cells (e.g. vascular endothelial cells) using methods of gene therapy, e.g. as described below.

In order to carry out the methods of the invention, one or more inhibitors or activators of Tsp42Ee, Tsp42El and/or GSK-3 expression or activity (e.g. ribozymes, antibodies, antisense molecules, small organic molecules, etc.) are administered to an individual to modulate a behavioral response to the consumption of alcohol and/or other substances of abuse. While this invention is described generally with reference to human subjects, veterinary applications are contemplated within the scope of this invention.

Various inhibitors may be administered, if desired, in the form of salts, esters, amides, prodrugs, derivatives, and the like, provided the salt, ester, amide, prodrug or derivative is suitable pharmacologically, i.e., effective in the present method. Salts, esters, amides, prodrugs and other derivatives of the active agents may be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

The active agents and various derivatives and/or formulations thereof are useful for parenteral, topical, oral, or local administration, such as by aerosol or transdermally, for prophylactic and/or therapeutic treatment of coronary disease and/or rheumatoid arthritis. The pharmaceutical compositions can be administered in a variety of unit dosage forms depending upon the method of administration. Suitable unit dosage forms, include, but are not limited to powders, tablets, pills, capsules, lozenges, suppositories, etc.

The active agent(s) and various derivatives and/or formulations thereof are typically combined with a pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers.

Other physiologically acceptable compounds include wetting agents, emulsifying agents, dispersing agents or preservatives which are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s). The excipients are preferably sterile and generally free of undesirable matter. These compositions may be sterilized by conventional, well known sterilization techniques.

The concentration of active agent(s) in the formulation can vary widely, and will be selected primarily based on fluid volumes, viscosities, body weight and the like in accordance with the particular mode of administration selected and the patient's needs.

In therapeutic applications, the compositions of this invention are administered to a patient suffering from a disease (e.g., atherosclerosis and/or associated conditions, and/or rheumatoid arthritis) in an amount sufficient to cure or at least partially arrest the disease and/or its symptoms (e.g. to reduce plaque formation, to reduce monocyte recruitment, etc.) An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the active agents of the formulations of this invention to effectively treat (ameliorate one or more symptoms) the patient.

In certain preferred embodiments, the Tsp42Ee, Tsp42El and/or GSK-3 modulators are administered orally (e.g. via a tablet) or as an injectable in accordance with standard methods well known to those of skill in the art. In other preferred embodiments, the Tsp42Ee, Tsp42El and/or GSK-3 modulators can also be delivered through the skin using conventional transdermal drug delivery systems, i.e., transdermal “patches” wherein the active agent(s) are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the active agent(s) and any other materials that are present.

The foregoing formulations and administration methods are intended to be illustrative and not limiting. It will be appreciated that, using the teaching provided herein, other suitable formulations and modes of administration can be readily devised.

### EXAMPLES

The following examples are offered to illustrate, but not to limit the claimed invention.

### Example 1

### A Tetraspanin Tsp42Ee Gene that Regulates Acute Ethanol-Induced Behaviors in Drosophila

A collection of Drosophila strains carrying single P-element insertions was screened for sensitivity to the activating effects of ethanol vapor in a locomotor tracking assay. One mutant with increased sensitivity was found to harbor a P-element insertion (5.10) near the gene Tsp42Ee, encoding putative tetraspanin, an integral membrane protein. This tetraspanin carries the following name according to the published Drosophila genome sequence: CG10106. A comparison of publicly available cDNA sequences and genomic sequences with that of the site of insertion of the P-element revealed that the P-element is inserted at the transcriptional start site of Tsp42Ee. We are generating antibodies to the proteins encoded by Tsp42Ee. We believe that Tsp42Ee and/or a mammalian homolog of Tsp42Ee provides a target for drugs to treat alcohol addiction and other drugs of abuse.

The line of interest was outcrossed for five generations and then tested and shown to retain its hyperactive phenotype. Inverse PCR was used to identify the site of insertion of the P-element 5.10. Sequencing of the PCR product confirmed that the P[GawB] insertion was at the site of transcription initiation for Tsp42Ee. The sequence of the open reading frame is: ATG GAC TGC GGC ACA TCT ATG GTC AAA TAC ATC CTC TTC ATA TTC AAC ACC ATT GTG TCG GTT ATC GGC ATC TTG GGC ATT GTT TAT GGC GTG CTG ATT CTG AAG AGC ATC GGT GTA GTT GAA GTT AAT GGA CAG GTG GGC TTC CCG ATA CAG GCT CTT ATG CCG ATC ATT CTT ATC AGC TTG GGC TCG ATT GTG GTC TTC ATT TCA TTC CTG GGA TGC TGC GGT GCC ATT CGC GAA TCC GTC TGC ATG ACC ATG AGC TAT GCC ACC TTC TTG CTG ATC CTG CTG ATC CTG CAG CTG ACG TTC GTT GTT CTG CTG TTT ACC CAC AGG GAA GAG TTT GAG AAC GCA ATG GGA AAC GTT ATC GAG AAT GCA TGG AAT TCT GAA CAT ACT TAT AAG GGA GGT GTC TTC GAC ACC ATT CAG AAA TCG TTG CAC TGC TGC GGA TCA AGC TCT GCT CTG GAC TAC ATC GGC AAG GGA GAC TTG GTG CCC CCA AGT TGT TGC AGC GGT TCG TGC CTG ATC CCG ACT AAC TAC TAC CCG GGA TGC CGT GGA AAG TTC GTC GAA TTA ATG ACC ACT GGA TCT GAT AAC GCT AAA TAT GTG GGC ATC GGC CTC ATC GGA ATA GAG CTG ATC GGC TTT ATC TTT GCC TGC TGC CTG GCC AAC AAC GTG CGT AAC TAC AAG CGC CGG AAC GCC TAC TAA (SEQ ID NO:2).

### Example 2

### A Tetraspanin Tsp42El Gene that Regulates Acute Ethanol-Induced Behaviors in Drosophila

A collection of Drosophila strains carrying single P-element insertions was screened for sensitivity to the activating effects of ethanol vapor in a locomotor tracking assay. One mutant with increased sensitivity was found to harbor a P-element insertion (4.43) near the gene Tsp42El, encoding a putative tetraspanin, an integral membrane protein. This tetraspanin carries the following name according to the published Drosophila genome sequence: CG12840. A comparison of publicly available cDNA sequences and genomic sequences with that of the site of insertion of the P-element revealed that the P-element is inserted at the transcriptional start site of Tsp42El. We are generating antibodies to the proteins encoded by Tsp42El. We believe that Tsp42El and/or a mammalian homolog of Tsp42El provides a target for drugs to treat alcohol addiction and other drugs of abuse.

The mutant of interest was outcrossed for five generations. Inverse PCR was used to identify the site of insertion of the P-element 4.43. Sequencing of the PCR product confirmed that the P[GawB] insertion was at the site of transcription initiation for Tsp42El. The sequence of open reading frame is: ATG GGT TGC GCA ACG GGC ACC ATA AAG TAC TCG CTG TTC CTG TTC AAT GCC TTA TGG GCG ATA CTC GGT ATC CTG GTG CTC ATC TTT GGC GGC CTT GGC TGG GGA GCA ATG CCA GAT GCA TAT GCC ATC GGC ATC TTA ATT CTG GGC GGT ACT ATC CTG GTA ATA TCC CTG TTT GGA TGC TGT GGA GCC GTT CGC GAA TCG CCG CGC ATG CTC TGG ACG TAT GCG TCA CTG CTG CTG ATT TTG CTG CTA CTT ATA GTG GCG TTT ATC ATC CTG AAT CCC AAA GAT GTA TTT AAA AAG TAC GCG CTT CAA ACG GTG GAG AAT CAG TGG GAG CTG GAG CAG ACG AAG CCT GGC AGT ATG GAT ATT ATT CAG AAA ACG TAC TAT TGC TGT GGC CGC GAC AGT GCC CAA GAC TAC TTG GAT ATC AAA TTC TGG AAC AAT ACC GTT CCA AGT AGC TGT TGC AAG GAC GAC AGC TGT GTG AAT CCA CTG AAT CTA TAT GTG CGC GGC TGC CTC ATC AAA GTG GAG GAG GCT TTT GCA GAT GAG GCA ACC ACT CTG GGC TAT TTG GAG TGG GGT CTG CTC GGA TTC AAC GCT GTC ATT CTA TTG CTG GCC ATC ATC TTG GCC ATT CAC TAC ACC AAC CGG CGG AGA CGA TAT AAC TAT TAG (SEQ ID NO:3).

### Example 3

### A Role For Glycogen Synthase Kinase-3 In Ethanol Induced Behavior

A collection of Drosophila strains carrying single P-element insertions was screened for sensitivity to the activating effects of ethanol vapor in a locomotor tracking assay. One mutant with increased sensitivity was found to harbor a P-element insertion (5.21) in the gene shaggy (sgg), encoding Glycogen Synthase Kinase 3 (GSK3), a protein serine/threonine kinase. This kinase carries the following name according to the published Drosophila genome sequence: CG2621. A comparison of publicly available cDNA sequences and genomic sequences with that of the site of insertion of the P-element revealed that the P-element is inserted in the second intron of the gene. We are currently generating antibodies to the protein encoded by sgg. We believe that sgg, or a that a mammalian homolog of sgg provides target for drugs to treat alcohol addiction and other drugs of abuse.

The mutant of interest was identified and the line of interest was outcrossed for five generations and then tested and shown to retain its hyperactive phenotype. Inverse PCR was used to identify the site of insertion of the P-element 5.21. Sequencing of the PCR product confirmed that the P[GawB] insertion was in the second intron of sgg. The sequence of the open reading frame is: ATG AGC GGT CGT CCA AGA ACT TCC TCC TTC GCC GAG GGC AAC AAA CAG TCG CCG AGT TTG GTG CTG GGC GGC GTC AAA ACA TGC AGT CGC GAT GGT TCT AAA ATC ACA ACA GTT GTT GCA ACA CCC GGC CAA GGC ACC GAT CGC GTA CAA GAG GTC TCC TAT ACA GAC ACA AAG GTC ATC GGC AAT GGC AGC TTC GGC GTC GTG TTC CAG GCA AAG CTC TGC GAT ACC GGC GAA CTG GTG GCA ATC AAA AAA GTT TTA CAA GAC AGA CGA TTT AAG AAT CGC GAA TTG CAA ATA ATG CGC AAA TTG GAG CAT TGT AAT ATT GTG AAG CTT TTG TAC TTT TTC TAT TCG AGT GGT GAA AAG CGT GAT GAA GTA TTT TTG AAT TTA GTC CTC GAA TAT ATA CCA GAA ACC GTA TAC AAA GTG GCT CGC CAA TAT GCC AAA ACC AAG CAA ACG ATA CCA ATC AAC TTT ATT CGG CTC TAC ATG TAT CAA CTG TTC AGA AGT TTG GCC TAC ATC CAC TCG CTG GGC ATT TGC CAT CGT GAT ATC AAG CCG CAG AAT CTT CTG CTC GAT CCG GAG ACG GCT GTG CTG AAG CTC TGT GAC TTT GGC AGC GCC AAA CAG CTG CTG CAC GGC GAG CCG AAT GTA TCG TAT ATC TGC TCC CGG TAT TAC CGC GCC CCC GAG CTC ATC TTT GGC GCC ATC AAT TAT ACA ACA AAG ATC GAT GTC TGG AGT GCC GGT TGC GTT TTG GCC GAA CTG CTG CTG GGC CAG CCC ATC TTC CCT GGC GAT TCC GGT GTG GAT CAG CTC GTC GAG GTC ATC AAG GTC CTG GGC ACA CCG ACA AGA GAA CAG ATA CGC GAA ATG AAT CCA AAC TAC ACG GAA TTC AAG TTC CCT CAG ATT AAG AGT CAT CCA TGG CAG AAA GTT TTC CGT ATA CGC ACT CCT ACA GAA GCT ATC AAC TTG GTG TCC CTG CTG CTC GAG TAT ACG CCC AGT GCC AGG ATC ACA CCG CTC AAG GCC TGC GCA CAT CCG TTC TTC GAT GAG CTA CGC ATG GAG GGT AAT CAC ACC TTG CCC AAC GGT CGC GAT ATG CCG CCG CTG TTC AAC TTC ACA GAG CAT GAG CTC TCA ATA CAG CCC AGC CTA GTG CCG CAG TTG TTG CCC AAG CAT CTG CAG AAC GCA TCC GGA CCT GGC GGC AAT CGA CCC TCG GCC GGC GGA GCA GCC TCC ATT GCG GCC AGC GGC TCC ACC AGC GTC TCG TCA ACG GGC AGT GGT GCC TCG GTG GAA GGA TCC GCC CAG CCA CAG TCG CAG GGT ACA GCA GCA GCT GCG GGA TCC GGA TCG GGC GGA GCA ACA GCA GGA ACC GGC GGA GCG AGT GCC GGT GGA CCC GGA TCT GGT AAC AAC AGT AGC AGC GGC GGA GCA TCG GGA GCG CCG TCC GCT GTG GCT GCC GGA GGA GCC AAT GCC GCC GTC GCT GGC GGT GCT GGT GGT GGT GGC GGA GCC GGT GCG GCG ACC GCA GCT GCA ACA GCA ACT GGC GCT ATA GGC GCG ACT AAT GCC GGC GGC GCC AAT GTA ACA GAT TCA TAG (SEQ ID NO:4)

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of identifying an agent that modulates a behavioral response to ethanol consumption, said method comprising:
contacting a cell or a tissue with a test agent; and
detecting expression or activity of a gene selected from the group consisting of a tetraspanin Tsp42Ee gene or a homologue or analogue thereof, a tetraspanin Tsp42El gene or a homologue or analogue thereof, and a Glycogen Synthase Kinase-3 gene or a homologue or analogue thereof;
wherein a change in activity or expression of said factor, as compared to a cell or tissue that is a control indicates that said test agent is a good candidate for modulating a behavioral response to ethanol consumption.

2. The method of claim 1, wherein said cell or tissue is a neural cell or tissue.

3. The method of claim 1, wherein said tetraspanin Tsp42Ee homologue or analogue is a human homologue or analogue.

4. The method of claim 1, wherein said tetraspanin Tsp42El homologue or analogue is a human homologue or analogue.

5. The method of claim 1, wherein said Glycogen Synthase Kinase-3 homologue or analogue is a human homologue or analogue.

6. The method of claim 1, wherein said control is a negative control comprising a cell or tissue contacted with said test agent at a lower concentration.

7. The method of claim 1, wherein said control is a negative control comprising a cell or tissue not contacted with said test agent.

8. The method of claim 1, wherein said control is a positive control comprising a cell or tissue contacted with said test agent at a higher concentration.

9. The method of claim 1, wherein said detecting comprises detecting a tetraspanin Tsp42Ee mRNA, a Tsp42El mRNA, or a Glycogen Synthase Kinase-3 mRNA.

10. The method of claim 9, wherein said level of tetraspanin Tsp42Ee mRNA, Tsp42El mRNA, or Glycogen Synthase Kinase-3 mRNA is measured by hybridizing said mRNA to a probe that specifically hybridizes to a tetraspanin Tsp42Ee nucleic acid, a Tsp42El nucleic acid, or a Glycogen Synthase Kinase-3 nucleic acid.

11. The method of claim 10, wherein said hybridizing is according to a method selected from the group consisting of a Northern blot, a Southern blot using DNA derived from the tetraspanin Tsp42Ee mRNA, Tsp42El mRNA, or Glycogen Synthase Kinase-3 mRNA, an array hybridization, an affinity chromatography, and an in situ hybridization.

12. The method of claim 10, wherein said probe is a member of a plurality of probes that forms an array of probes.

13. The method of claim 9, wherein the level of tetraspanin Tsp42Ee mRNA, Tsp42El mRNA, or Glycogen Synthase Kinase-3 mRNA is measured using a nucleic acid amplification reaction.

14. The method of claim 1, wherein said detecting comprises detecting a tetraspanin Tsp42Ee protein, a Tsp42El protein, and/or Glycogen Synthase Kinase-3 protein.

15. The method of claim 14, wherein said detecting is via a method selected from the group consisting of capillary electrophoresis, a Western blot, mass spectroscopy, ELISA, immunochromatography, and immunohistochemistry.

16. The method of claim 1, wherein said cell is cultured ex vivo.

17. The method of claim 1, wherein said test agent is contacted to a mammal comprising said cell or tissue.

18. A method of prescreening for an agent that modulates a behavioral response to ethanol consumption, said method comprising:
contacting a gene or gene product from a gene selected from the group consisting of a tetraspanin Tsp42Ee gene or a homologue or analogue thereof, a tetraspanin Tsp42El gene or a homologue or analogue thereof, and a Glycogen Synthase Kinase-3 gene or a homologue or analogue thereof with a test agent; and
detecting specific binding of said test agent to said gene or gene product, wherein specific binding indicates that said agent is a candidate modulator of a behavioral response to ethanol consumption.

19. The method of claim 17, wherein said homologue or analogue is a human homologue or analogue.

20. The method of claim 17, further comprising recording test agents that specifically bind to said gene or gene product, in a database of candidate agents that modulate an organisms behavioral response to ethanol consumption.

21. The method of claim 17, wherein said test agent is not an antibody.

22. The method of claim 17, wherein said test agent is not a protein.

23. The method of claim 17, wherein said test agent is not a nucleic acid.

24. The method of claim 17, wherein said test agent is a small organic molecule.

25. The method of claim 17, wherein said detecting comprises detecting specific binding of said test agent to a tetraspanin Tsp42Ee nucleic acid, and/or to a Tsp42El nucleic acid, and/or to a Glycogen Synthase Kinase-3 nucleic acid.

26. The method of claim 25, wherein said binding is detected using a method selected from the group consisting of a Northern blot, a Southern blot using DNA derived from a tetraspanin Tsp42Ee gene, a tetraspanin Tsp42El gene, and/or a Glycogen Synthase Kinase-3 gene, an array hybridization, an affinity chromatography, and an in situ hybridization.

27. The method of claim 17, wherein said detecting comprises detecting specific binding of said test agent to a tetraspanin Tsp42Ee protein, and/or to a Tsp42El protein, and/or to a Glycogen Synthase Kinase-3 protein.

28. The method of claim 27, wherein said detecting is via a method selected from the group consisting of capillary electrophoresis, a Western blot, mass spectroscopy, ELISA, immunochromatography, and immunohistochemistry.

29. The method of claim 17, wherein said test agent is contacted directly to the gene or gene product.

30. The method of claim 17, wherein said test agent is contacted to a cell containing gene or gene product.

31. A method of altering the behavioral response of an organism to ethanol consumption, said method comprising altering expression or activity of a gene selected from the group consisting of a tetraspanin Tsp42Ee gene or a homologue or analogue thereof, a tetraspanin Tsp42El gene or a homologue or analogue thereof, and a Glycogen Synthase Kinase-3 gene or a homologue or analogue thereof.

32. The method of claim 34, wherein said altering comprises increasing the expression or activity of said gene.

33. The method of claim 34, wherein said altering comprises decreasing the expression or activity of said gene.

34. An antibody that specifically binds to a gene product from a gene selected from the group consisting of a tetraspanin Tsp42Ee gene or a homologue or analogue thereof, a tetraspanin Tsp42El gene or a homologue or analogue thereof, and a Glycogen Synthase Kinase-3 gene or a homologue or analogue thereof.

35. The antibody of claim 5, wherein said antibody is a monoclonal antibody.

36. The antibody of claim 5, wherein said antibody is a polyclonal antibody.

37. The antibody of claim 5, wherein said antibody is a single chain antibody.

38. A knockout animal, said mammal comprising a disruption in an endogenous gene selected from the group consisting of a tetraspanin Tsp42Ee gene or a homologue or analogue thereof, a tetraspanin Tsp42El gene or a homologue or analogue thereof, and a Glycogen Synthase Kinase-3 gene or a homologue or analogue thereof.

39. The animal of claim 38, wherein said animal shows an altered response to consumption of alcohol or other substances of abuse as compared to a wild-type animal.

40. The animal of claim 39, wherein said animal is a mammal.

41. The mammal of claim 40, wherein the mammal is selected from the group consisting of an equine, a bovine, a rodent, a porcine, a lagomorph, a feline, a canine, a murine, a caprine, an ovine, and a non-human primate.

42. The mammal of claim 40, wherein the disruption is selected from the group consisting of an insertion, a deletion, a frameshift mutation, a substitution, and a stop codon.

43. The mammal of claim 42, wherein the disruption comprises an insertion of an expression cassette into the endogenous gene.

44. The mammal of claim 43, wherein said expression cassette comprises a selectable marker.

45. The mammal of claim 44, wherein the expression cassette comprises a neomycin phosphotransferase gene operably linked to at least one regulatory element.

46. The mammal of claim 40, wherein said disruption is in a somatic cell.

47. The mammal of claim 40, wherein said disruption is in a germ cell.

48. The mammal of claim 40, wherein the mammal is homozygous for the disrupted gene.

49. The mammal of claim 40, wherein the mammal is heterozygous for the disrupted gene.


SEQUENCE LISTING (4 sequences)

SEQ ID NO: 1 - RNA, 14 nt, Artificial
  Synthetic ribozyme.
nnnbngucnnnnnn

SEQ ID NO: 2 - DNA, 687 bp, Drosophila sp.
atggactgcggcacatctatggtcaaatacatcctcttcatattcaacaccattgtgtcggttatcggcatcttgggcattgtttatggcgtgctgattctgaagagcatcggtgtagttgaagttaatggacaggtgggcttcccgatacaggctcttatgccgatcattcttatcagcttgggctcgattgtggtcttcatttcattcctgggatgctgcggtgccattcgcgaatccgtctgcatgaccatgagctatgccaccttcttgctgatcctgctgatcctgcagctgacgttcgttgttctgctgtttacccacagggaagagtttgagaacgcaatgggaaacgttatcgagaatgcatggaattctgaacatacttataagggaggtgtcttcgacaccattcagaaatcgttgcactgctgcggatcaagctctgctctggactacatcggcaagggagacttggtgcccccaagttgttgcagcggttcgtgcctgatcccgactaactactacccgggatgccgtggaaagttcgtcgaattaatgaccactggatctgataacgctaaatatgtgggcatcggcctcatcggaatagagctgatcggctttatctttgcctgctgcctggccaacaacgtgcgtaactacaagcgccggaacgcctactaa

SEQ ID NO: 3 - DNA, 654 bp, Drosophila sp.
atgggttgcgcaacgggcaccataaagtactcgctgttcctgttcaatgccttatgggcgatactcggtatcctggtgctcatctttggcggccttggctggggagcaatgccagatgcatatgccatcggcatcttaattctgggcggtactatcctggtaatatccctgtttggatgctgtggagccgttcgcgaatcgccgcgcatgctctggacgtatgcgtcactgctgctgattttgctgctacttatagtggcgtttatcatcctgaatcccaaagatgtatttaaaaagtacgcgcttcaaacggtggagaatcagtgggagctggagcagacgaagcctggcagtatggatattattcagaaaacgtactattgctgtggccgcgacagtgcccaagactacttggatatcaaattctggaacaataccgttccaagtagctgttgcaaggacgacagctgtgtgaatccactgaatctatatgtgcgcggctgcctcatcaaagtggaggaggcttttgcagatgaggcaaccactctgggctatttggagtggggtctgctcggattcaacgctgtcattctattgctggccatcatcttggccattcactacaccaaccggcggagacgatataactattag

SEQ ID NO: 4 - DNA, 1545 bp, Drosophila sp.
atgagcggtcgtccaagaacttcctccttcgccgagggcaacaaacagtcgccgagtttggtgctgggcggcgtcaaaacatgcagtcgcgatggttctaaaatcacaacagttgttgcaacacccggccaaggcaccgatcgcgtacaagaggtctcctatacagacacaaaggtcatcggcaatggcagcttcggcgtcgtgttccaggcaaagctctgcgataccggcgaactggtggcaatcaaaaaagttttacaagacagacgatttaagaatcgcgaattgcaaataatgcgcaaattggagcattgtaatattgtgaagcttttgtactttttctattcgagtggtgaaaagcgtgatgaagtatttttgaatttagtcctcgaatatataccagaaaccgtatacaaagtggctcgccaatatgccaaaaccaagcaaacgataccaatcaactttattcggctctacatgtatcaactgttcagaagtttggcctacatccactcgctgggcatttgccatcgtgatatcaagccgcagaatcttctgctcgatccggagacggctgtgctgaagctctgtgactttggcagcgccaaacagctgctgcacggcgagccgaatgtatcgtatatctgctcccggtattaccgcgcccccgagctcatctttggcgccatcaattatacaacaaagatcgatgtctggagtgccggttgcgttttggccgaactgctgctgggccagcccatcttccctggcgattccggtgtggatcagctcgtcgaggtcatcaaggtcctgggcacaccgacaagagaacagatacgcgaaatgaatccaaactacacggaattcaagttccctcagattaagagtcatccatggcagaaagttttccgtatacgcactcctacagaagctatcaacttggtgtccctgctgctcgagtatacgcccagtgccaggatcacaccgctcaaggcctgcgcacatccgttcttcgatgagctacgcatggagggtaatcacaccttgcccaacggtcgcgatatgccgccgctgttcaacttcacagagcatgagctctcaatacagcccagcctagtgccgcagttgttgcccaagcatctgcagaacgcatccggacctggcggcaatcgaccctcggccggcggagcagcctccattgcggccagcggctccaccagcgtctcgtcaacgggcagtggtgcctcggtggaaggatccgcccagccacagtcgcagggtacagcagcagctgcgggatccggatcgggcggagcaacagcaggaaccggcggagcgagtgccggtggacccggatctggtaacaacagtagcagcggcggagcatcgggagcgccgtccgctgtggctgccggaggagccaatgccgccgtcgctggcggtgctggtggtggtggcggagccggtgcggcgaccgcagctgcaacagcaactggcgctataggcgcgactaatgccggcggcgccaatgtaacagattcatag